SEQUENCE LISTING (52 sequences)

SEQ ID NO: 1 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 2 - DNA, 4140 bp, Artificial sequence
  Yarrowia optimized Cas9
atggacaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaa

SEQ ID NO: 3 - protein, 9 aa, Simian virus 40
MAPKKKRKV

SEQ ID NO: 4 - DNA, 546 bp, Yarrowia lipolytica
tcgacgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacaccatg

SEQ ID NO: 5 - DNA, 4683 bp, artificial sequence
  Yarrowia optimized Cas9 expression cassette
tcgacgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacaccatggacaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaa

SEQ ID NO: 6 - DNA, 10706 bp, artificial sequence
  pZufCas9CS
catggacaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaagcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgcgtacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaatcgatttcgacagtaattaattaagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacac

SEQ ID NO: 7 - DNA, 4144 bp, artificial sequence
  Cas9-SV40 fusion
acaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaagcggccgc

SEQ ID NO: 8 - DNA, 35 bp, Artificial sequence
  Cas9 forward primer
gggggaattcgacaagaaatactccatcggcctgg

SEQ ID NO: 9 - DNA, 31 bp, Artificial sequence
  Cas9 reverse primer
ccccaagcttagcggccgcttagacctttcg

SEQ ID NO: 10 - DNA, 4166 bp, Artificial sequence
  Cas9 PCR product
gggggaattcgacaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaagcggccgctaagcttgggg

SEQ ID NO: 11 - DNA, 4092 bp, Artificial sequence
  pBAD/HisB
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggggttctcatcatcatcatcatcatggtatggctagcatgactggtggacagcaaatgggtcgggatctgtacgacgatgacgataaggatccgagctcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 12 - DNA, 8237 bp, Artificial sequence
  pRF48
aattcgacaagaaatactccatcggcctggacattggaaccaactctgtcggctgggctgtcatcaccgacgagtacaaggtgccctccaagaaattcaaggtcctcggaaacaccgatcgacactccatcaagaaaaacctcattggtgccctgttgttcgattctggcgagactgccgaagctaccagactcaagcgaactgctcggcgacgttacacccgacggaagaaccgaatctgctacctgcaggagatcttttccaacgagatggccaaggtggacgattcgttctttcatcgactggaggaatccttcctcgtcgaggaagacaagaaacacgagcgtcatcccatctttggcaacattgtggacgaggttgcttaccacgagaagtatcctaccatctaccacctgcgaaagaaactcgtcgattccaccgacaaggcggatctcagacttatctacctcgctctggcacacatgatcaagtttcgaggtcatttcctcatcgagggcgatctcaatcccgacaacagcgatgtggacaagctgttcattcagctcgttcagacctacaaccagctgttcgaggaaaaccccatcaatgcctccggagtcgatgcaaaggccatcttgtctgctcgactctcgaagagcagacgactggagaacctcattgcccaacttcctggcgagaaaaagaacggactgtttggcaacctcattgccctttctcttggtctcacacccaacttcaagtccaacttcgatctggcggaggacgccaagctccagctgtccaaggacacctacgacgatgacctcgacaacctgcttgcacagattggcgatcagtacgccgacctgtttctcgctgccaagaacctttcggatgctattctcttgtctgacattctgcgagtcaacaccgagatcacaaaggctcccctttctgcctccatgatcaagcgatacgacgagcaccatcaggatctcacactgctcaaggctcttgtccgacagcaactgcccgagaagtacaaggagatctttttcgatcagtcgaagaacggctacgctggatacatcgacggcggagcctctcaggaagagttctacaagttcatcaagccaattctcgagaagatggacggaaccgaggaactgcttgtcaagctcaatcgagaggatctgcttcggaagcaacgaaccttcgacaacggcagcattcctcatcagatccacctcggtgagctgcacgccattcttcgacgtcaggaagacttctacccctttctcaaggacaaccgagagaagatcgagaagattcttacctttcgaatcccctactatgttggtcctcttgccagaggaaactctcgatttgcttggatgactcgaaagtccgaggaaaccatcactccctggaacttcgaggaagtcgtggacaagggtgcctctgcacagtccttcatcgagcgaatgaccaacttcgacaagaatctgcccaacgagaaggttcttcccaagcattcgctgctctacgagtactttacagtctacaacgaactcaccaaagtcaagtacgttaccgagggaatgcgaaagcctgccttcttgtctggcgaacagaagaaagccattgtcgatctcctgttcaagaccaaccgaaaggtcactgttaagcagctcaaggaggactacttcaagaaaatcgagtgtttcgacagcgtcgagatttccggagttgaggaccgattcaacgcctctttgggcacctatcacgatctgctcaagattatcaaggacaaggattttctcgacaacgaggaaaacgaggacattctggaggacatcgtgctcactcttaccctgttcgaagatcgggagatgatcgaggaacgactcaagacatacgctcacctgttcgacgacaaggtcatgaaacaactcaagcgacgtagatacaccggctggggaagactttcgcgaaagctcatcaacggcatcagagacaagcagtccggaaagaccattctggactttctcaagtccgatggctttgccaaccgaaacttcatgcagctcattcacgacgattctcttaccttcaaggaggacatccagaaggcacaagtgtccggtcagggcgacagcttgcacgaacatattgccaacctggctggttcgccagccatcaagaaaggcattctccagactgtcaaggttgtcgacgagctggtgaaggtcatgggacgtcacaagcccgagaacattgtgatcgagatggccagagagaaccagacaactcaaaagggtcagaaaaactcgcgagagcggatgaagcgaatcgaggaaggcatcaaggagctgggatcccagattctcaaggagcatcccgtcgagaacactcaactgcagaacgagaagctgtatctctactatctgcagaatggtcgagacatgtacgtggatcaggaactggacatcaatcgtctcagcgactacgatgtggaccacattgtccctcaatcctttctcaaggacgattctatcgacaacaaggtccttacacgatccgacaagaacagaggcaagtcggacaacgttcccagcgaagaggtggtcaaaaagatgaagaactactggcgacagctgctcaacgccaagctcattacccagcgaaagttcgacaatcttaccaaggccgagcgaggcggtctgtccgagctcgacaaggctggcttcatcaagcgtcaactcgtcgagaccagacagatcacaaagcacgtcgcacagattctcgattctcggatgaacaccaagtacgacgagaacgacaagctcatccgagaggtcaaggtgattactctcaagtccaaactggtctccgatttccgaaaggactttcagttctacaaggtgcgagagatcaacaattaccaccatgcccacgatgcttacctcaacgccgtcgttggcactgcgctcatcaagaaataccccaagctcgaaagcgagttcgtttacggcgattacaaggtctacgacgttcgaaagatgattgccaagtccgaacaggagattggcaaggctactgccaagtacttcttttactccaacatcatgaactttttcaagaccgagatcaccttggccaacggagagattcgaaagagaccacttatcgagaccaacggcgaaactggagagatcgtgtgggacaagggtcgagactttgcaaccgtgcgaaaggttctgtcgatgcctcaggtcaacatcgtcaagaaaaccgaggttcagactggcggattctccaaggagtcgattctgcccaagcgaaactccgacaagctcatcgctcgaaagaaagactgggatcccaagaaatacggtggcttcgattctcctaccgtcgcctattccgtgcttgtcgttgcgaaggtcgagaagggcaagtccaaaaagctcaagtccgtcaaggagctgctcggaattaccatcatggagcgatcgagcttcgagaagaatcccatcgacttcttggaagccaagggttacaaggaggtcaagaaagacctcattatcaagctgcccaagtactctctgttcgaactggagaacggtcgaaagcgtatgctcgcctccgctggcgagctgcagaagggaaacgagcttgccttgccttcgaagtacgtcaactttctctatctggcttctcactacgagaagctcaagggttctcccgaggacaacgaacagaagcaactcttcgttgagcagcacaaacattacctcgacgagattatcgagcagatttccgagttttcgaagcgagtcatcctggctgatgccaacttggacaaggtgctctctgcctacaacaagcatcgggacaaacccattcgagaacaggcggagaacatcattcacctgtttactcttaccaacctgggtgctcctgcagctttcaagtacttcgataccactatcgaccgaaagcggtacacatccaccaaggaggttctcgatgccaccctgattcaccagtccatcactggcctgtacgagacccgaatcgacctgtctcagcttggtggcgactccagagccgatcccaagaaaaagcgaaaggtctaagcggccgctaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggggttctcatcatcatcatcatcatggtatggctagcatgactggtggacagcaaatgggtcgggatctgtacgacgatgacgataaggatccgagctcgagatctgcagctggtaccatatggg

SEQ ID NO: 13 - DNA, 23 bp, Escherichia coli
  misc_feature (1)..(23): GalK-1 target site
atcagcggcaatgtgccgcaggg

SEQ ID NO: 14 - DNA, 23 bp, Escherichia coli
  misc_feature (1)..(23): GalK-2 target site
atgaccggcggcggatttggcgg

SEQ ID NO: 15 - DNA, 23 bp, Escherichia coli
  misc_feature (1)..(23): GalK-3 target site
atagttttcatgtgcgacaatgg

SEQ ID NO: 16 - DNA, 23 bp, Escherichia coli
  misc_feature (1)..(23): GalK-4 target site
atgatctttcttgccgagcgcgg

SEQ ID NO: 17 - DNA, 80 bp, Streptococcus pyogenes
gttttagagctagaaatagcaagttaaaataaggctagtccgttatcaacttgaaaaagtggcaccgagtcggtgctttt

SEQ ID NO: 18 - DNA, 100 bp, Artificial sequence
  GalK-1 sgRNA template
atcagcggcaatgtgccgcagttttagagctagaaatagcaagttaaaataaggctagtccgttatcaacttgaaaaagtggcaccgagtcggtgctttt

SEQ ID NO: 19 - DNA, 100 bp, Artificial sequence
  GalK-2 sgRNA template
atgaccggcggcggatttgggttttagagctagaaatagcaagttaaaataaggctagtccgttatcaacttgaaaaagtggcaccgagtcggtgctttt

SEQ ID NO: 20 - DNA, 100 bp, Artificial sequence
  GalK-3 sgRNA template
atagttttcatgtgcgacaagttttagagctagaaatagcaagttaaaataaggctagtccgttatcaacttgaaaaagtggcaccgagtcggtgctttt

SEQ ID NO: 21 - DNA, 100 bp, Artificial sequence
  GalK-4 sgRNA template
atgatctttcttgccgagcggttttagagctagaaatagcaagttaaaataaggctagtccgttatcaacttgaaaaagtggcaccgagtcggtgctttt

SEQ ID NO: 22 - RNA, 100 nt, Artificial sequence
  GalK-1 sgRNA
aucagcggcaaugugccgcaguuuuagagcuagaaauagcaaguuaaaauaaggcuaguccguuaucaacuugaaaaaguggcaccgagucggugcuuuu

SEQ ID NO: 23 - RNA, 100 nt, Artificial sequence
  GalK-2 sgRNA
augaccggcggcggauuuggguuuuagagcuagaaauagcaaguuaaaauaaggcuaguccguuaucaacuugaaaaaguggcaccgagucggugcuuuu

SEQ ID NO: 24 - RNA, 100 nt, Artificial sequence
  GalK-3 sgRNA
auaguuuucaugugcgacaaguuuuagagcuagaaauagcaaguuaaaauaaggcuaguccguuaucaacuugaaaaaguggcaccgagucggugcuuuu

SEQ ID NO: 25 - RNA, 100 nt, Artificial sequence
  GalK-4 sgRNA
augaucuuucuugccgagcgguuuuagagcuagaaauagcaaguuaaaauaaggcuaguccguuaucaacuugaaaaaguggcaccgagucggugcuuuu

SEQ ID NO: 26 - DNA, 52 bp, bacteriophage lambda
ggttatctctggcggtgttgacataaataccactggcggtgatactgagcac

SEQ ID NO: 27 - DNA, 43 bp, bacteriophage lambda
gttaataacaggcctgctggtaatcgcaggcctttttattttt

SEQ ID NO: 28 - DNA, 212 bp, Artificial sequence
  GalK-1 sgRNA expression cassette
gggaagcttggttatctctggcggtgttgacataaataccactggcggtgatactgagcacatcagcggcaatgtgccgcagttttagagctagaaatagcaagttaaaataaggctagtccgttatcaacttgaaaaagtggcaccgagtcggtggtgcgttaataacaggcctgctggtaatcgcaggcctttttatttttggatccggg

SEQ ID NO: 29 - DNA, 212 bp, Artificial sequence
  GalK-2 sgRNA expression cassette
gggaagcttggttatctctggcggtgttgacataaataccactggcggtgatactgagcacatgaccggcggcggatttgggttttagagctagaaatagcaagttaaaataaggctagtccgttatcaacttgaaaaagtggcaccgagtcggtggtgcgttaataacaggcctgctggtaatcgcaggcctttttatttttggatccggg

SEQ ID NO: 30 - DNA, 212 bp, Artificial sequence
  GalK-3 sgRNA expression cassette
gggaagcttggttatctctggcggtgttgacataaataccactggcggtgatactgagcacatagttttcatgtgcgacaagttttagagctagaaatagcaagttaaaataaggctagtccgttatcaacttgaaaaagtggcaccgagtcggtggtgcgttaataacaggcctgctggtaatcgcaggcctttttatttttggatccggg

SEQ ID NO: 31 - DNA, 212 bp, Artificial sequence
  GalK-4 sgRNA expression cassette
gggaagcttggttatctctggcggtgttgacataaataccactggcggtgatactgagcacatgatctttcttgccgagcggttttagagctagaaatagcaagttaaaataaggctagtccgttatcaacttgaaaaagtggcaccgagtcggtggtgcgttaataacaggcctgctggtaatcgcaggcctttttatttttggatccggg

SEQ ID NO: 32 - DNA, 4245 bp, Artificial sequence
  pACYC184
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 33 - DNA, 4099 bp, Artificial sequence
  pRF50
gatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggttatctctggcggtgttgacataaataccactggcggtgatactgagcacatcagcggcaatgtgccgcagttttagagctagaaatagcaagttaaaataaggctagtccgttatcaacttgaaaaagtggcaccgagtcggtggtgcgttaataacaggcctgctggtaatcgcaggcctttttatttttg

SEQ ID NO: 34 - DNA, 4099 bp, artificial sequence
  pRF51
agcttggttatctctggcggtgttgacataaataccactggcggtgatactgagcacatgaccggcggcggatttgggttttagagctagaaatagcaagttaaaataaggctagtccgttatcaacttgaaaaagtggcaccgagtcggtggtgcgttaataacaggcctgctggtaatcgcaggcctttttatttttggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgata

SEQ ID NO: 35 - DNA, 4099 bp, artificial sequence
  pRF53
gatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggttatctctggcggtgttgacataaataccactggcggtgatactgagcacatagttttcatgtgcgacaagttttagagctagaaatagcaagttaaaataaggctagtccgttatcaacttgaaaaagtggcaccgagtcggtggtgcgttaataacaggcctgctggtaatcgcaggcctttttatttttg

SEQ ID NO: 36 - DNA, 4099 bp, Artificial sequence
  pRF55
agcttggttatctctggcggtgttgacataaataccactggcggtgatactgagcacatgatctttcttgccgagcggttttagagctagaaatagcaagttaaaataaggctagtccgttatcaacttgaaaaagtggcaccgagtcggtggtgcgttaataacaggcctgctggtaatcgcaggcctttttatttttggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgata

SEQ ID NO: 37 - DNA, 454 bp, Escherichia coli
  misc_feature (1)..(454): 454bp 5′ galK
ggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaa

SEQ ID NO: 38 - DNA, 29 bp, Artificial sequence
  5′ forward primer
gggaagcttggattatgttcagcgcgagc

SEQ ID NO: 39 - DNA, 40 bp, Artificial sequence
  5′ reverse primer
tgccagtgcgggagtttcgtttcttacactccggattcgc

SEQ ID NO: 40 - DNA, 483 bp, Artificial sequence
  upstream overlap extension product
gggaagcttggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaacgaaactcccgcactggca

SEQ ID NO: 41 - DNA, 376 bp, Escherichia coli
  misc_feature (1)..(376): 376bp 3′ galK
acgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgc

SEQ ID NO: 42 - DNA, 40 bp, Artificial sequence
  3′ forward primer
gcgaatccggagtgtaagaaacgaaactcccgcactggca

SEQ ID NO: 43 - DNA, 30 bp, Artificial sequence
  3′ reverse primer
gggaagcttgcaaacagcacctgacgatcg

SEQ ID NO: 44 - DNA, 405 bp, Artificial Sequence
  downstream overlap extension product
gcgaatccggagtgtaagaaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgcaagcttccc

SEQ ID NO: 45 - DNA, 848 bp, Artificial sequence
  galK deletion polynucleotide modification template
gggaagcttggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgcaagcttccc

SEQ ID NO: 46 - DNA, 2804 bp, Artificial sequence
  pKD3
agattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 47 - DNA, 2458 bp, Artificial sequence
  pRF113
agctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgca

SEQ ID NO: 48 - DNA, 1717 bp, Escherichia coli
  misc_feature (1)..(1717): galK locus
ggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaatgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctgaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgc

SEQ ID NO: 49 - DNA, 21 bp, artificial sequence
  GalK forward
ggcgaagagaatcaacactgg

SEQ ID NO: 50 - DNA, 21 bp, Artificial sequence
  GalK Reverse
gcaaacagcacctgacgatcg

SEQ ID NO: 51 - DNA, 1136 bp, Escherichia coli
  misc_feature (1)..(1136): galK deletion
ggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgcggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaagaaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgc

SEQ ID NO: 52 - DNA, 23 bp, unknown
  Example of a Cas9 target sitePAM sequence
  misc_feature (1)..(20): n = A, C, T, or G
  misc_feature (21)..(21): n = A, C, T, or G (indicated as an “X” in Specification)
nnnnnnnnnnnnnnnnnnnnngg

(19) United States
(12) Patent Application Publication
(10) Pub. No.: US 2017/0369866 A1
(43) Pub. Date: Dec. 28, 2017

(54) COMPOSITIONS AND METHODS FOR EFFICIENT GENE EDITING IN E. COLI USING GUIDE RNA/CAS ENDONUCLEASE SYSTEMS IN COMBINATION WITH CIRCULAR POLYNUCLEOTIDE MODIFICATION TEMPLATES

(71) Applicant: E I DU PONT DE NEMOURS AND COMPANY, Wilmington, DE (US)

(72) Inventors: Ryan L. Frisch, Newark, DE (US); Ethel N. Jackson, Greenville, DE (US)

(73) Assignee: E I DU PONT DE NEMOURS AND COMPANY, Wilmington, DE (US)

(21) Appl. No.: 15/533,475

(22) PCT Filed: Dec. 2, 2015
(86) PCT No.: PCT/US2015/063434

§ 371 Date: Jun. 6, 2017

Related U.S. Application Data
(60) Provisional application No. 62/092,914, filed on Dec. 17, 2014.

(51) Int. Cl. C12N 15/10; C12N 15/82; C12N 9/22; C12N 15/70; C12N 15/90; C12N 15/113

(52) U.S. Cl. CPC C12N 15/102; C12N 15/70; C12N 15/10; C12N 15/902; C12N 15/8213; C12N 9/22; C12N 15/113; C12N 2320/10

(57) ABSTRACT

Compositions and methods are provided for genome modification of a target sequence in the genome of an Escherichia coli cell. The methods and compositions employ a guide RNA/Cas endonuclease system in combination with a circular polynucleotide modification template to provide an effective system for editing target sites within the genome of an Escherichia coli cell.

This application claims the benefit of U.S. Provisional Application No. 62/092914 filed Dec. 17, 2014, incorporated herein in its entirety by reference.

### FIELD OF INVENTION

The invention relates to the field of bacterial molecular biology, in particular, to compositions and methods for editing a nucleotide sequence in the genome of Escherichia coli.

### REFERENCE TO SEQUENCE LISTING SUBMITTED ELECTRONICALLY

The official copy of the sequence listing is submitted electronically via EFS-Web as an ASCII formatted sequence listing with a file named 20151117_CL6256PCT_ST25.txt created on Nov. 17, 2015, and having a size of 106 kilobytes and is filed concurrently with the specification. The sequence listing contained in this ASCII-formatted document is part of the specification and is herein incorporated by reference in its entirety.

### BACKGROUND

A way to understand the function of a gene within an organism is to inhibit its expression. Inhibition of gene expression can be accomplished, for example, by interrupting or deleting the DNA sequence of the gene, resulting in “knock-out” of the gene (Austin et al., Nat. Genetics 36:921-924). Gene knock-outs mostly have been carried out through homologous recombination (HR), a technique applicable across a wide array of organisms from bacteria to mammals. Another way for studying gene function can be through genetic “knock-in”, which is also usually performed by HR. HR for gene targeting has been shown to be enhanced when the targeted DNA site contains a double-strand break (Rudin et al., Genetics 122:519-534; Smih et al., Nucl. Acids Res. 23:5012-5019). Strategies for introducing double-strand breaks to facilitate HR-mediated DNA targeting have therefore been developed. For example, zinc finger nucleases have been engineered to cleave specific DNA sites leading to enhanced levels of HR at the site when a polynucleotide modification template DNA was present (Bibikova et al., Science 300:764; Bibikova et al., Mol. Cell. Biol. 21:289-297). Similarly, artificial meganucleases (homing endonucleases) and transcription activator-like effector (TALE) nucleases have also been developed for use in HR-mediated DNA targeting (Epinat et al., Nucleic Acids Res. 31: 2952-2962; Miller et al., Nat. Biotech. 29:143-148).

Loci encoding CRISPR (clustered regularly interspaced short palindromic repeats) DNA cleavage systems have been found exclusively in about 40% of bacterial genomes and most archaeal genomes (Horvath and Barrangou, Science 327:167-170; Karginov and Hannon, Mol. Cell 37:7-19). In particular, the CRISPR-associated (Cas) RNA-guided endonuclease (RGEN), Cas9, of the type II CRIPSR system has been developed as a means for introducing site-specific DNA strand breaks that stimulate HR (U.S. Provisional Appl. No. 61/868,706, filed August 22, 2013). The sequence of the RNA component of Cas9 can be designed such that Cas9 recognizes and cleaves DNA containing (i) sequence complementary to a portion of the RNA component and (ii) a protospacer adjacent motif (PAM) sequence.

Native RNA/Cas9 complexes comprise two RNA sequences, a CRISPR RNA (crRNA) and a trans-activating CRISPR RNA (tracrRNA). A crRNA contains, in the 5′-to-3′ direction, a unique sequence complementary to a target DNA site and a portion of a sequence encoded by a repeat region of the CRISPR locus from which the crRNA was derived. A tracrRNA contains, in the 5′-to-3′ direction, a sequence that anneals with the repeat region of crRNA and a stem loop-containing portion. Recent work has led to the development of guide RNAs (gRNA), which are chimeric sequences containing, in the 5′-to-3′ direction, a crRNA linked to a tracrRNA (U.S. patent application Ser. No. 14/463,687, filed Aug. 20, 2014).

Recombinant DNA technology has made it possible to modify DNA sequences in the genome of an organism, thus, altering the organism's phenotype. Although several approaches have been developed to target a specific site for modification in the genome of an organism such as E. coli, there still remains a need for more efficient and effective methods for editing a nucleotide sequence in the genome of an Escherichia coli cell.

### SUMMARY

The present disclosure includes compositions and methods for genome modification of a target sequence in the genome of an Escherichia coli cell. The methods and compositions employ a guide RNA/Cas endonuclease system (also referred to as an RGEN) in combination with a circular polynucleotide modification template to provide an effective system for editing target sites within the genome of an Escherichia coli cell. The methods and compositions also employ a guide RNA/Cas endonuclease system in combination with a circular donor DNA to provide an effective system for gene knock-in an Escherichia coli cell.

In one embodiment of the disclosure , the method comprises a method for editing a nucleotide sequence in the genome of an Escherichia coli cell, the method comprising providing at least one recombinant DNA construct comprising a DNA sequence encoding a guide RNA and a circular polynucleotide modification template to an E.coli cell comprising a Cas9 endonuclease DNA sequence operably linked to an inducible promoter, wherein said Cas9 endonuclease DNA sequence encodes a Cas9 endonuclease that is capable of introducing a double-strand break at a target site in the genome of said E. coli cell, wherein said polynucleotide modification template comprises at least one nucleotide modification of said nucleotide sequence. The nucleotide sequence in the genome of an E. coli cell can be selected from the group consisting of a promoter sequence, a terminator sequence, a regulatory element sequence, a coding sequence, a prophage, a pseudogene, an exogenous gene, an endogenous gene. The recombinant DNA construct comprising a DNA sequence encoding a guide RNA can be provided via a circular plasmid. The recombinant DNA construct and the circular polynucleotide modification template can each be provided on separate plasm ids. The recombinant DNA construct and the circular polynucleotide modification template can be provided on a single plasmid. The recombinant DNA construct and the circular polynucleotide template can be provided via one mean selected from the group consisting of electroporation, heat-shock, phage delivery, mating, conjugation and transduction. The target site in the genome of the E. coli cell, can be flanked by a first genomic region and a second genomic region, wherein the circular polynucleotide template further comprises a first region of homology to said first genomic region and a second region of homology to said second genomic region.

In one embodiment, the E. coli cell does not express an exogenous recombinase protein, a RecET protein, a lambda-red protein, or a RecBCD inhibitor.

In one embodiment of the disclosure, the method comprises a method for producing a galK mutant E.coli cell, the method comprising: a) providing at least one circular recombinant DNA construct comprising a DNA sequence encoding a guide RNA and at least one circular polynucleotide modification template to an E. coli cell comprising a Cas9 endonuclease DNA sequence operably linked to an inducible promoter, wherein said Cas9 endonuclease DNA sequence encodes a Cas endonuclease that is capable of introducing a double-strand break at a target site within a galK genomic sequence in the E. coli genome, wherein said circular polynucleotide modification template comprises at least one nucleotide modification of said galK genomic sequence; b) growing progeny cells from the E. coli cell of (a); c) evaluating the progeny cells of (b) for the presence of said at least one nucleotide modification.

In one embodiment of the disclosure, the method comprises a method for editing a nucleotide sequence in the genome of an Escherichia coli cell, the method comprising providing at least a first recombinant DNA construct comprising a DNA sequence encoding a guide RNA, a circular polynucleotide modification template, and a second recombinant DNA construct comprising a DNA sequence encoding Cas9 endonuclease operably linked to an inducible promoter, to an E.coli cell, wherein the Cas9 endonuclease introduces a double-strand break at a target site in the genome of said E. coli cell, wherein said polynucleotide modification template comprises at least one nucleotide modification of said nucleotide sequence. The first recombinant DNA construct, the second recombinant DNA construct, and the circular polynucleotide modification template can each be provided on separate plasmids. The first recombinant DNA construct, the second recombinant DNA construct, and the circular polynucleotide modification template can be provided on a single plasmid

### BRIEF DESCRIPTION OF THE DRAWINGS AND SEQUENCES

FIG. 1. Use of a circular plasmid (template plasmid) comprising a polynucleotide modification template for gene editing of a native target in E. coli cells comprising a Cas9 plasmid. The schematic illustrates an E. coli cell containing a native target to be edited (located in the E. coli target genome) and a Cas9 plasmid comprising a Cas9 expression cassette driven by an inducible promoter (for example, Pbad). The polynucleotide modification template containing the desired edit (shown by a white star) to the native target sequence (shown as a black bar) flanked by two homologous regions (HR1 and HR2, allowing homologous recombination) is provided to E. coli cells (of which the Cas9 endonuclease expression was induced) via the template plasmid, together with a guide RNA plasmid comprising the guide RNA expression cassette capable of expressing a guide RNA (gRNA). The induced E. coli cell are capable of expressing the Cas9 endonuclease and form a guide RNA/Cas9 endonuclease complex (also referred to as RGEN) that is capable of mediating cleavage of the native target sequence allowing for homologous recombination mediated gene editing.

FIG. 2. Use of a circular plasmid (template plasmid) comprising a polynucleotide modification template for gene editing of a native target in E. coli cells lacking a Cas9 plasmid. The schematic illustrates an E. coli cell containing a native target sequence to be edited (located in the E. coli target genome) promoter. The polynucleotide modification template containing the desired edit (shown by a white star) to the native target sequence (shown as a black bar) flanked by two homologous regions (HR1 and HR2, allowing homologous recombination) is provided to E. coli cells via the template plasmid, together with a guide RNA plasmid (comprising the guide RNA expression cassette) and a Cas9 plasmid (comprising an inducible Cas9 expression cassette driven by a Pbad). Once the E. coli cell are induced, the induced cells are capable of expressing the Cas9 endonuclease and form a guide RNA/Cas9 endonuclease complex (also referred to as RGEN) that is capable of mediating cleavage of the native target sequence allowing for homologous recombination mediated gene editing.

FIG. 3 shows a single guide polynucleotide containing a Cas endonuclease recognition domain (CER) domain (black) linked to the variable targeting domain (VT) (grey).

FIG. 4 shows an SDS-PAGE gel of Cas9 expression from pRF48 in E. coli cells pre and post induction with arabinose. Marker weights are indicated in kilodaltons (kDa). The band corresponding to Cas9 in the gel is indicated (Cas9).

FIG. 5 illustrates the galK gene of E. coli (black). The four native target sites in galK are indicated by arrows labelled with the target site name and the direction of the arrow indicating forward or reverse strand of the target DNA.

FIG. 6 shows an agarose gel of DNA from a colony PCR of the galK locus of galactose resistant E. coli after gene editing with the guide RNA/Cas9 endonuclease complex (RGEN). Each lane corresponds to an individual galactose resistant colony. Marker weights are given in kilobases (kb). The size of the desired edit (deletion) is indicated next to the band. The size of the unedited allele is also indicated (WT). Two control reactions (WT and pRF113) are run on the gel to indicate the WT and edited allele respectively.

TABLE 1
Summary of Nucleic Acid and Protein SEQ ID Numbers

| | | Protein |
|---|---|---|
| | Nucleic acid | SEQ ID |
| Description | SEQ ID NO. | NO. |
| Streptococcus pyogenes Cas9 open reading | 1 | |
| frame | (4107 bases) | |
| Streptococcus pyogenes Cas9 including | 2 | |
| C-terminal linker and SV40 NLS | (4140 bases) | |
| (“Cas9-NLS”); open reading frame | | |
| codon-optimized for expression in Y. lipolytica. | | |
| Simian virus40 NLS | | 3 |
| | | (9 aa) |
| Yarrowia lipolytica FBA1 promoter. | 4 | |
| | (546 bases) | |
| Yarrowia optimized Cas9 expression cassette | 5 | |
| | (4683 bases) | |
| pZUFCas9 plasmid. | 6 | |
| | (10706 bases) | |
| Cas9-SV40 fusion | 7 | |
| | (4144 bases) | |
| Cas9-NLS forward PCR primer. | 8 | |
| | (35 bases) | |
| Cas9-NLS reverse PCR primer. | 9 | |
| | (31 bases) | |
| EcoRI-Cas9-NLS-HinDIII PCR product | 10 | |
| | (4166 bases) | |
| pBAD/HisB plasmid | 11 | |
| | (4092 bases) | |
| pRF48 plasmid | 12 | |
| | (8237 bases) | |
| GalK-1 target site | 13 | |
| | (23 bases) | |
| GalK-2 target site | 14 | |
| | (23 bases) | |
| GalK-3 target site | 15 | |
| | (23 bases) | |
| GalK-4 target site | 16 | |
| | (23 bases) | |
| Cas9 recognition domain (CER)25 | 17 | |
| | (80 bases) | |
| GalK-1 sgRNA template DNA | 18 | |
| | (100 bases) | |
| GalK-2 sgRNA template DNA | 19 | |
| | (100 bases) | |
| GalK-3 sgRNA template DNA | 20 | |
| | (100 bases) | |
| GalK-4sgRNA template DNA | 21 | |
| | (100 bases) | |
| GalK-1 sgRNA | 22 | |
| | (100 bases) | |
| GalK-2 sgRNA | 23 | |
| | (100 bases) | |
| GalK-3 sgRNA | 24 | |
| | (100 bases) | |
| GalK-4sgRNA | 25 | |
| | (100 bases) | |
| Lambda PL promoter | 26 | |
| | (52 bases) | |
| Lambda Terminator | 27 | |
| | (43 bases) | |
| GalK-1 sgRNA expression cassette | 28 | |
| | (212 bases) | |
| GalK-1 sgRNA expression cassette | 29 | |
| | (212 bases) | |
| GalK-1 sgRNA expression cassette | 30 | |
| | (212 bases) | |
| GalK-1 sgRNA expression cassette | 31 | |
| | (212 bases) | |
| pACYC184 | 32 | |
| | (4245 bases) | |
| pRF50 | 33 | |
| | (4099 bases) | |
| pRF51 | 34 | |
| | (4099 bases) | |
| pRF53 | 35 | |
| | (4099 bases) | |
| pRF55 | 36 | |
| | (4099 bases) | |
| 454bp 5′ galK | 37 | |
| | (454 bases) | |
| 5′ forward primer | 38 | |
| | (29 bases) | |
| 5′ reverse primer | 39 | |
| | (40 bases) | |
| upstream overlap extension product | 40 | |
| | (483 bases) | |
| 376bp 3′ galK | 41 | |
| | (376 bases) | |
| 3′ forward primer | 42 | |
| | (40 bases) | |
| 3′ reverse primer | 43 | |
| | (30 bases) | |
| downstream overlap extension product | 44 | |
| | (405 bases) | |
| galK deletion polynucleotide modification | 45 | |
| template | (848 bases) | |
| pKD3 | 46 | |
| | (2804 bases) | |
| pRF113; | 47 | |
| | (2458 bases) | |
| galK locus | 48 | |
| | (1717 bases) | |
| GalK forward primer | 49 | |
| | (21 bases) | |
| GalK Reverse primer | 50 | |
| | (21 bases) | |
| galK deletion locus (amplified from the edited | 51 | |
| strains) | (1136 bases) | |
| Example of a Cas9 target site:PAM sequence | 52 | |
| | (23 bases) | |

### DETAILED DESCRIPTION

The disclosures of all cited patent and non-patent literature are incorporated herein by reference in their entirety.

As used herein, the term “disclosure” or “disclosed disclosure” is not meant to be limiting, but applies generally to any of the disclosures defined in the claims or described herein. These terms are used interchangeably herein.

Compositions and methods are provided for genome modification of a target sequence in the genome of an Escherichia coli cell. The methods and compositions employ a guide RNA/Cas endonuclease system in combination with a circular polynucleotide modification template to provide an effective system for editing target sites within the genome of an Escherichia coli cell.

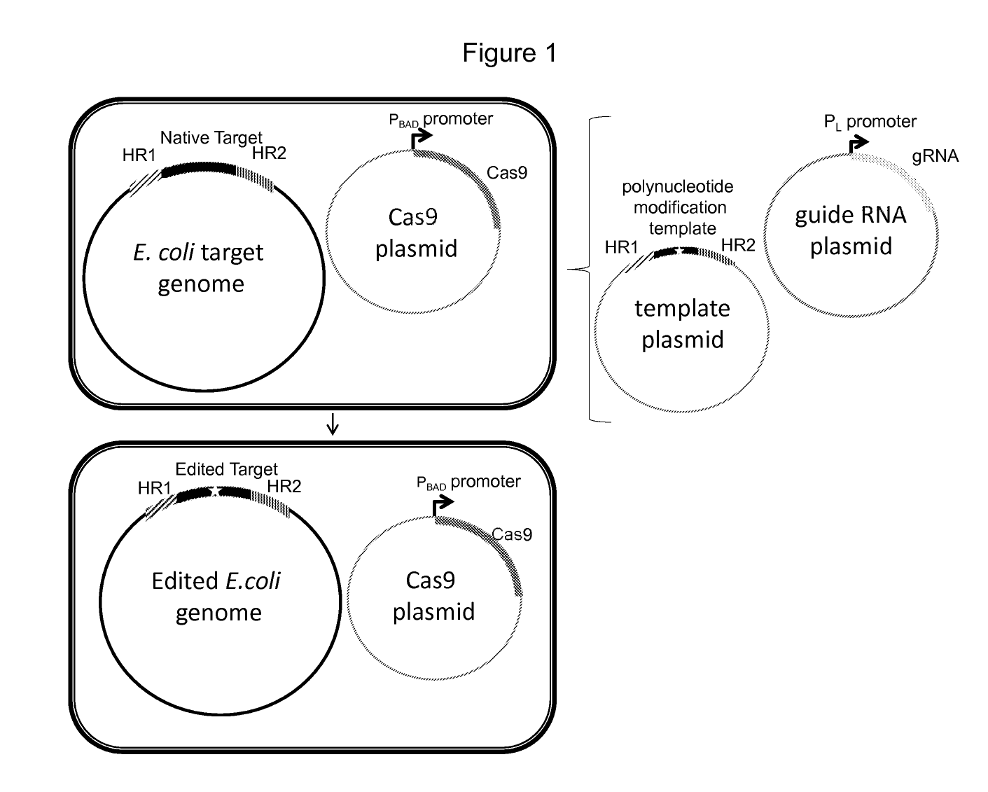

The use of a circular plasmid (template plasmid) comprising a polynucleotide modification template for gene editing of a native target in E. coli cells comprising a Cas9 plasmid is illustrated in FIG. 1 and described herein. The schematic illustrates an E. coli cell containing a native target to be edited (located in the E. coli target genome) and a Cas9 plasmid comprising a Cas9 expression cassette driven by an inducible promoter (for example, Pbad). The polynucleotide modification template containing the desired edit (shown by a white star) to the native target sequence (shown as a black bar) flanked by two homologous regions (HR1 and HR2, allowing homologous recombination) is provided to E. coli cells (of which the Cas9 endonuclease expression was induced) via the template plasmid, together with a guide RNA plasmid comprising the guide RNA expression cassette capable of expressing a guide RNA (gRNA). The induced E. coli cell are capable of expressing the Cas9 endonuclease and form a guide RNA/Cas9 endonuclease complex (also referred to as RGEN) that is capable of mediating cleavage of the native target sequence allowing for homologous recombination mediated gene editing.

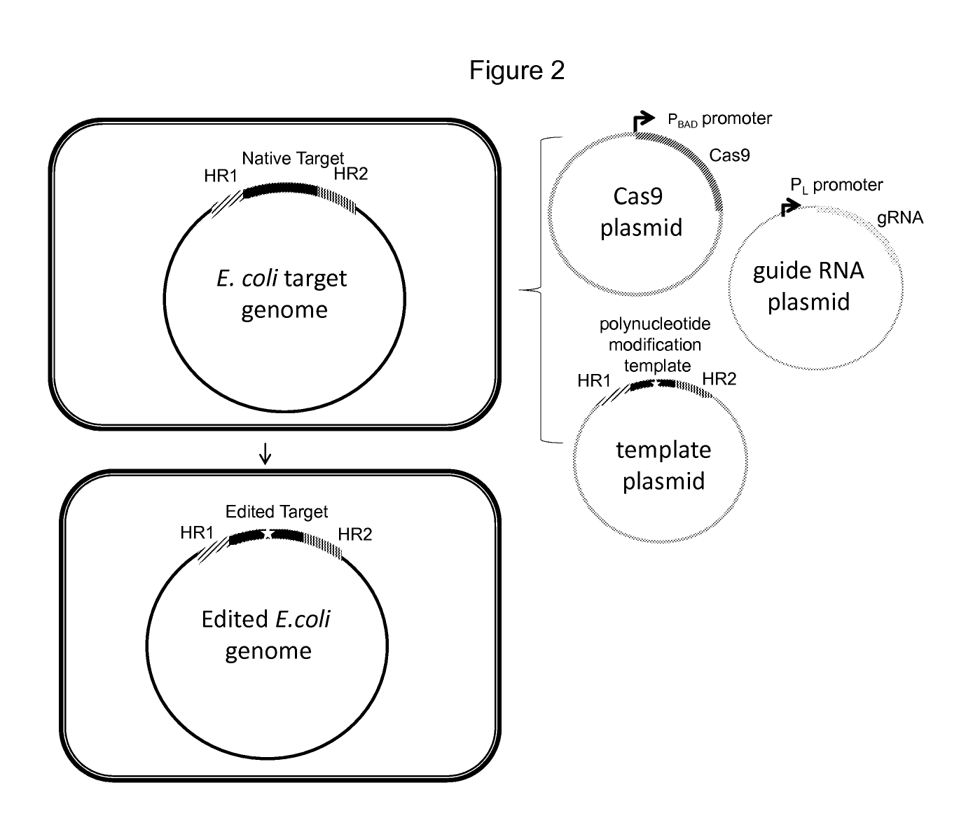

The use of a circular plasmid (template plasmid) comprising a polynucleotide modification template for gene editing of a native target in E. coli cells lacking a Cas9 plasmid is illustrated in FIG. 2 and described herein. The schematic illustrates an E. coli cell containing a native target sequence to be edited (located in the E. coli target genome). The polynucleotide modification template containing the desired edit (shown by a white star) to the native target sequence (shown as a black bar) flanked by two homologous regions (HR1 and HR2, allowing homologous recombination) is provided to E. coli cells via the template plasmid, together with a guide RNA plasmid (comprising the guide RNA expression cassette) and a Cas9 plasmid (comprising an inducible Cas9 expression cassette driven by a Pbad). Once the E. coli cell are induced , the induced cells are capable of expressing the Cas9 endonuclease and form a guide RNA/Cas9 endonuclease complex (also referred to as RGEN) that is capable of mediating cleavage of the native target sequence allowing for homologous recombination mediated gene editing.

A circular plasmid comprising a donor DNA comprising a polynucleotide of interest can also be used for a for gene knock-in E. coli as described herein.

The term “CRISPR” (clustered regularly interspaced short palindromic repeats) refers to certain genetic loci encoding factors of class I, II, or III DNA cleavage systems, for example, used by bacterial and archaeal cells to destroy foreign DNA (Horvath and Barrangou, Science 327:167-170). Components of CRISPR systems are taken advantage of herein in a heterologous manner for DNA targeting in cells.

The terms “type II CRISPR system” and “type II CRISPR-Cas system” are used interchangeably herein and refer to a DNA cleavage system utilizing a Cas9 endonuclease in complex with at least one RNA component. For example, a Cas9 can be in complex with a CRISPR RNA (crRNA) and a trans-activating CRISPR RNA (tracrRNA). In another example, a Cas9 can be in complex with a guide RNA. Thus, crRNA, tracrRNA, and guide RNA are non-limiting examples of RNA components herein.

The term CRISPR-associated (“Cas”) endonuclease herein refers to a Cas protein encoded by a Cas gene. A Cas endonuclease, when in complex with a suitable RNA component, is capable of cleaving all or part of a specific DNA target sequence. For example, it is capable of introducing a double-strand break in a specific DNA target sequence; it can alternatively be characterized as being able to cleave one or both strands of a specific DNA target sequence. A Cas endonuclease can unwind the DNA duplex at the target sequence and cleaves at least one DNA strand, as mediated by recognition of the target sequence by a crRNA or guide RNA that is in complex with the Cas. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. A preferred Cas protein herein is Cas9.

“Cas9” (formerly referred to as Cas5, Csn1, or Csx12) herein refers to a Cas endonuclease of a type II CRISPR system that forms a complex with crRNA and tracrRNA, or with a guide RNA, for specifically recognizing and cleaving all or part of a DNA target sequence. Cas9 protein comprises an RuvC nuclease domain and an HNH (H-N-H) nuclease domain, each of which cleaves a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). “Apo-Cas9” refers to Cas9 that is not complexed with an RNA component. Apo-Cas9 can bind DNA, but does so in a non-specific manner, and cannot cleave DNA (Sternberg et al., Nature 507:62-67).

The term “CRISPR RNA” (crRNA) herein refers to an RNA sequence that can form a complex with one or more Cas proteins (e.g., Cas9) and provides DNA binding specificity to the complex. A crRNA provides DNA binding specificity since it contains a “variable targeting domain” (VT) that is complementary to a strand of a DNA target sequence. A crRNA further comprises a “repeat sequence” (“tracr RNA mate sequence”) encoded by a repeat region of the CRISPR locus from which the crRNA was derived. A repeat sequence of a crRNA can anneal to sequence at the 5′-end of a tracrRNA. crRNA in native CRISPR systems is derived from a “pre-crRNA” transcribed from a CRISPR locus. A pre-crRNA comprises spacer regions and repeat regions; spacer regions contain unique sequence complementary to a DNA target site sequence. Pre-crRNA in native systems is processed to multiple different crRNAs, each with a guide sequence along with a portion of repeat sequence. CRISPR systems utilize crRNA, for example, for DNA targeting specificity.

The term “trans-activating CRISPR RNA” (tracrRNA) herein refers to a non-coding RNA used in type II CRISPR systems, and contains, in the 5′-to-3′ direction, (i) a sequence that anneals with the repeat region of CRISPR type II crRNA and (ii) a stem loop-containing portion (Deltcheva et al., Nature 471:602-607).

A “CRISPR DNA” (crDNA) can optionally be used instead of an RNA component. A crDNA has a DNA sequence corresponding to the sequence of a crRNA as disclosed herein. A crDNA can be used with a tracrRNA in a crDNA/tracrRNA complex, which in turn can be associated with an RGEN protein component. U.S. Appl. No. 61/953,090 discloses crDNA and the methods of its use in RGEN-mediated DNA targeting. It is contemplated that any disclosure herein regarding a crRNA can similarly apply to using a crDNA, accordingly. Thus, in embodiments herein incorporating a crDNA, an “RNA-guided endonuclease” (RGEN) could instead be referred to as a complex comprising at least one Cas protein and at least one crDNA.

As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (an RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization.

A guide polynucleotide that solely comprises ribonucleic acids is also referred to as a “guide RNA”. The guide RNA can form a complex with a Cas endonuclease referred to a guide RNA/Cas endonuclease complex (also referred to as an RGEN) The terms “guide RNA” (gRNA) and “single guide RNA” (sgRNA) are used interchangeably herein. A gRNA herein can refer to a chimeric sequence containing a crRNA operably linked to a tracrRNA. Alternatively, a gRNA can refer to a synthetic fusion of a crRNA and a tracrRNA, for example. A gRNA can also be characterized in terms of having a variable targeting domain followed by Cas endonuclease recognition (CER) domain. A CER domain can comprise a tracrRNA mate sequence followed by a tracrRNA sequence.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain (“crNucleotide”) is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides).

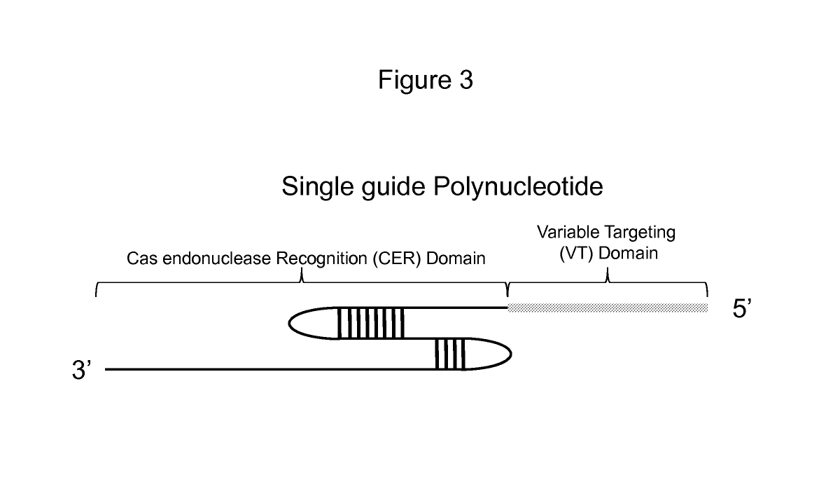

The guide polynucleotide can also be a single molecule comprising a first nucleotide sequence domain (referred to as Variable Targeting domain or VT domain, FIG. 3) that is complementary to a nucleotide sequence in a target DNA and a second nucleotide domain (referred to as Cas endonuclease recognition domain or CER domain, FIG. 3) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise an RNA sequence, a DNA sequence, or a, RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising an RNA sequence, a DNA sequence, or an RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides).

Thus, a guide polynucleotide and a type II Cas endonuclease in certain embodiments can form a complex with each other (referred to as a “guide polynucleotide/Cas endonuclease complex” or also referred to as “guide polynucleotide/Cas endonuclease system”), wherein the guide polynucleotide/Cas endonuclease complex can direct the Cas endonuclease to target a genomic target site in a cell (e.g., plant cell), optionally enabling the Cas endonuclease to introduce a single- or double-strand break into the genomic target site. A guide polynucleotide/Cas endonuclease complex can be linked to at least one CPP, wherein such complex is capable of binding to, and optionally creating a single- or double-strand break to, a target site of a cell (e.g., a plant cell).

The term “variable targeting domain” or “VT domain” is used interchangeably herein and refers to a nucleotide sequence that is complementary to one strand (nucleotide sequence) of a double strand DNA target site. The percent complementation between the first nucleotide sequence domain (VT domain) and the target sequence can be at least 50%, 51° A, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, an RNA sequence, a modified DNA sequence, a modified RNA sequence (see, e.g., modifications described herein), or any combination thereof.

The term “Cas endonuclease recognition domain” or “CER domain” of a guide polynucleotide is used interchangeably herein and relates to a nucleotide sequence (such as a second nucleotide sequence domain of a guide polynucleotide), that interacts with a Cas endonuclease polypeptide. A CER domain can be composed of a DNA sequence, an RNA sequence, a modified DNA sequence, a modified RNA sequence (see, e.g., modifications described herein), or any combination thereof.

The term “RNA-guided endonuclease”, “RGEN”, “guide RNA/Cas endonuclease complex”, “guide RNA/Cas endonuclease system” can be used interchangeably herein and refers to a complex comprising at least one CRISPR (clustered regularly interspaced short palindromic repeats)-associated (Cas) protein and at least one RNA component. The terms “protein component of an RGEN” and “RGEN protein component” are used interchangeably herein and refer to a Cas protein, which is, or forms part of, the endonuclease component of an RGEN. A protein component in certain embodiments can be a complete endonuclease (e.g., Cas9); such a protein component can alternatively be referred to as “the endonuclease component” of an RGEN. An RGEN herein typically has specific DNA targeting activity, given its association with at least one RNA component.

The term “RNA component” herein refers to an RNA component of an RGEN containing a ribonucleic acid sequence that is complementary to a strand of a DNA target sequence. This complementary sequence is referred to herein as a “guide sequence” or “variable targeting domain” sequence (FIG. 3). Examples of suitable RNA components herein include crRNA and guide RNA. RNA components in certain embodiments (e.g., guide RNA alone, crRNA+tracrRNA) can render an RGEN competent for specific DNA targeting.

Briefly, an RNA component of an RGEN contains sequence that is complementary to a DNA sequence in a target site sequence. Based on this complementarity, an RGEN can specifically recognize and cleave a particular DNA target site sequence. An RGEN herein can comprise Cas protein(s) and suitable RNA component(s) of any of the four known CRISPR systems (Horvath and Barrangou, Science 327:167-170) such as a type I, II, or III CRISPR system. An RGEN in preferred embodiments comprises a Cas9 endonuclease (CRISPR II system) and at least one RNA component (e.g., a crRNA and tracrRNA, or a gRNA).

An RGEN protein component can refer to a Cas protein such as Cas9. Examples of suitable Cas proteins include one or more Cas endonucleases of type I, II, or III CRISPR systems (Bhaya et al., Annu. Rev. Genet. 45:273-297, incorporated herein by reference). A type I CRISPR Cas protein can be a Cas3 or Cas4 protein, for example. A type II CRISPR Cas protein can be a Cas9 protein, for example. A type III CRISPR Cas protein can be a Cas10 protein, for example. A Cas9 protein is used in certain preferred embodiments. A Cas protein in certain embodiments may be a bacterial or archaeal protein. Type I-III CRISPR Cas proteins herein are typically prokaryotic in origin; type I and III Cas proteins can be derived from bacterial or archaeal species, whereas type II Cas proteins (i.e., a Cas9) can be derived from bacterial species, for example. In other embodiments, suitable Cas proteins include one or more of Cas1, Cas1B, Cas2, Cas3, Cas4, Cas5, Cas6, Cas7, Cas8, Cas9, Cas10, Csyl, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In other aspects of the disclosed disclosure, a Cas protein herein can be from any of the following genera: Aeropyrum, Pyrobaculum, Sulfolobus, Archaeoglobus, Haloarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Streptococcus, Treponema, Francisella, or Thermotoga. Alternatively, a Cas protein herein can be encoded, for example, by any of SEQ ID NOs:462-465, 467-472, 474-477, 479-487, 489-492, 494-497, 499-503, 505-508, 510-516, or 517-521 as disclosed in U.S. Appl. Publ. No. 2010/0093617, which is incorporated herein by reference.

An RGEN protein component can comprise a Cas9 amino acid sequence, for example. An RGEN comprising this type of protein component typically can be characterized as having Cas9 as the endonuclease component of the RGEN. The amino acid sequence of a Cas9 protein herein, as well as certain other Cas proteins herein, may be derived from a Streptococcus (e.g., S. pyogenes, S. pneumoniae, S. thermophilus, S. agalactiae, S. parasanguinis, S. oralis, S. salivarius, S. macacae, S. dysgalactiae, S. anginosus, S. constellatus, S. pseudoporcinus, S. mutans), Listeria (e.g., L. innocua), Spiroplasma (e.g., S. apis, S. syrphidicola), Peptostreptococcaceae, Atopobium, Porphyromonas (e.g., P. catoniae), Prevotella (e.g., P. intermedia), Veillonella, Treponema (e.g., T. socranskii, T. denticola), Capnocytophaga, Finegoldia (e.g., F. magna), Coriobacteriaceae (e.g., C. bacterium), Olsenella (e.g., O. profusa), Haemophilus (e.g., H. sputorum, H. pittmaniae), Pasteurella (e.g., P. bettyae), Olivibacter (e.g., O. sitiensis), Epilithonimonas (e.g., E. tenax), Mesonia (e.g., M. mobilis), Lactobacillus, Bacillus (e.g., B. cereus), Aquimarina (e.g., A. muelleri), Chryseobacterium (e.g., C. palustre), Bacteroides (e.g., B. graminisolvens), Neisseria (e.g., N. meningitidis), Francisella (e.g., F. novicida), or Flavobacterium (e.g., F. frigidarium, F. soli) species, for example. An S. pyogenes Cas9 is preferred in certain aspects herein. As another example, a Cas9 protein can be any of the Cas9 proteins disclosed in Chylinski et al. (RNA Biology 10:726-737), which is incorporated herein by reference.

Accordingly, the sequence of a Cas9 protein herein can comprise, for example, any of the Cas9 amino acid sequences disclosed in GenBank Accession Nos. G3ECR1 (S. thermophilus), WP_026709422, WP_027202655, WP_027318179, WP_027347504, WP_027376815, WP_027414302, WP_027821588, WP_027886314, WP_027963583, WP_028123848, WP_028298935, Q03JI6 (S. thermophilus), EGP66723, EGS38969, EGV05092, EHI65578 (S. pseudoporcinus), EIC75614 (S. oralis), EID22027 (S. constellatus), EIJ69711, EJP22331 (S. oralis), EJP26004 (S. anginosus), EJP30321, EPZ44001 (S. pyogenes), EPZ46028 (S. pyogenes), EQL78043 (S. pyogenes), EQL78548 (S. pyogenes), ERL10511, ERL12345, ERL19088 (S. pyogenes), ESA57807 (S. pyogenes), ESA59254 (S. pyogenes), ESU85303 (S. pyogenes), ETS96804, UC75522, EGR87316 (S. dysgalactiae), EGS33732, EGV01468 (S. oralis), EHJ52063 (S. macacae), EID26207 (S. oralis), EID33364, EIG27013 (S. parasanguinis), EJF37476, EJO19166 (Streptococcus sp. BS35b), EJU16049, EJU32481, YP_006298249, ERF61304, ERK04546, ETJ95568 (S. agalactiae), TS89875, ETS90967 (Streptococcus sp. SR4), ETS92439, EUB27844 (Streptococcus sp. BS21), AFJ08616, EUC82735 (Streptococcus sp. CM6), EWC92088, EWC94390, EJP25691, YP_008027038, YP_008868573, AGM26527, AHK22391, AHB36273, Q927P4, G3ECR1, or Q99ZW2 (S. pyogenes), which are incorporated by reference. A variant of any of these Cas9 protein sequences may be used, but should have specific binding activity, and optionally cleavage or nicking activity, toward DNA when associated with an RNA component herein. Such a variant may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of the reference Cas9.

Alternatively, a Cas9 protein herein can be encoded by the SEQ ID NOs: 1-2, for example. Alternatively still, a Cas9 protein may comprise an amino acid sequence that is at least about 80%, 81° A, 82%, 83%, 84%, 85%, 86%, 87%, 88%, 89%, 90%, 91° A, 92%, 93%, 94%, 95%, 96%, 97%, 98%, or 99% identical to any of the foregoing amino acid sequences, for example. Such a variant Cas9 protein should have specific binding activity, and optionally cleavage or nicking activity, toward DNA when associated with an RNA component herein.

The origin of a Cas protein used herein (e.g., Cas9) may be from the same species from which the RNA component(s) is derived, or it can be from a different species. For example, an RGEN comprising a Cas9 protein derived from a Streptococcus species (e.g., S. pyogenes or S. thermophilus) may be complexed with at least one RNA component having a sequence (e.g., crRNA repeat sequence, tracrRNA sequence) derived from the same Streptococcus species. Alternatively, the origin of a Cas protein used herein (e.g., Cas9) may be from a different species from which the RNA component(s) is derived (the Cas protein and RNA component(s) may be heterologous to each other); such heterologous Cas/RNA component RGENs should have DNA targeting activity.

Determining binding activity and/or endonucleolytic activity of a Cas protein herein toward a specific target DNA sequence may be assessed by any suitable assay known in the art, such as disclosed in U.S. Pat. No. 8,697,359, which is disclosed herein by reference. A determination can be made, for example, by expressing a Cas protein and suitable RNA component in a cell, and then examining the predicted DNA target site for the presence of an indel (a Cas protein in this particular assay would typically have complete endonucleolytic activity [double-strand cleaving activity]). Examining for the presence of an alteration/modification (e.g., indel) at the predicted target site could be done via a DNA sequencing method or by inferring alteration/modification formation by assaying for loss of function of the target sequence, for example.

In still another example, Cas protein activity can be determined using an in vitro assay in which a Cas protein and suitable RNA component are mixed together along with a DNA polynucleotide containing a suitable target sequence. This assay can be used to detect binding (e.g., gel-shift) by Cas proteins lacking cleavage activity, or cleavage by Cas proteins that are endonucleolytically competent.

A Cas protein herein such as a Cas9 can further comprise a heterologous nuclear localization sequence (NLS) in certain aspects. A heterologous NLS amino acid sequence herein may be of sufficient strength to drive accumulation of a Cas protein, or Cas protein-CPP complex, in a detectable amount in the nucleus of a cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. Non-limiting examples of suitable NLS sequences herein include those disclosed in U.S. Pat. Nos. 6,660,830 and 7,309,576 (e.g., Table 1 therein), which are both incorporated herein by reference. A Cas protein as disclosed herein can be fused with a CPP (an example of a Cas protein covalently linked to a CPP), for example. It would be understood that such a Cas-CPP fusion protein can also comprise an NLS as described above. It would also be understood that, in embodiments in which a Cas protein is fused with an amino acid sequence targeting a different organelle (e.g., mitochondria), such a Cas protein typically would not contain an NLS.

A Cas protein can be part of a fusion protein comprising one or more heterologous protein domains (e.g., 1, 2, 3, or more domains in addition to the Cas protein). For example, a Cas protein can be covalently linked to a CPP and/or one or more additional heterologous amino acid sequences (see U.S. provisional patent application NO. 62/036652, filed Aug. 13, 2014). A Cas protein can also be covalently linked to one or more additional heterologous amino acid sequences not including a CPP, for example (a CPP would be non-covalently linked to a Cas fusion protein in such embodiments). A fusion protein comprising a Cas protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His, poly-histidine], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein in other embodiments may be in fusion with a protein that binds DNA molecules or other molecules, such as maltose binding protein (MBP), S-tag, Lex A DNA binding domain (DBD), GAL4A DNA binding domain, and herpes simplex virus (HSV) VP16. Additional domains that may be part of a fusion protein comprising a Cas protein herein are disclosed in U.S. Patent Appl. Publ. No. 2011/0059502, which is incorporated herein by reference. In certain embodiments in which a Cas protein is fused to a heterologous protein (e.g., a transcription factor), the Cas protein has DNA recognition and binding activity (when in complex with a suitable RNA component herein), but no DNA nicking or cleavage activity.

Other examples of heterologous domains that can be linked to a Cas protein herein include amino acid sequences targeting the protein to a particular organelle (i.e., localization signal). Examples of organelles that can be targeted include mitochondria and chloroplasts. Typically, such targeting domains are used instead of an NLS when targeting extra-nuclear DNA sites. A mitochondrial targeting sequence (MTS) can be situated at or near the N-terminus of a Cas protein, for example. MTS examples are disclosed in U.S. Patent Appl. Publ. Nos. 2007/0011759 and 2014/0135275, which are incorporated herein by reference. A chloroplast targeting sequence can be as disclosed in U.S. Patent Appl. Publ. No. 2010/0192262 or 2012/0042412, for example, which are incorporated herein by reference.

The protein component of an RGEN can be associated with at least one RNA component (thereby constituting a complete RGEN) that comprises a sequence complementary to a target site sequence on a chromosome or episome in a cell, for example. The RGEN in such embodiments can bind to the target site sequence, and optionally cleave one or both DNA strands at the target site sequence. An RGEN can cleave one or both strands of a DNA target sequence, for example. An RGEN can cleave both strands of a DNA target sequence in another example. It would be understood that in all these embodiments, an RGEN protein component can be covalently or non-covalently linked to at least one CPP in an RGEN protein-CPP complex. The association of an RGEN protein-CPP complex with an RNA component herein can be characterized as forming an RGEN-CPP complex. Any disclosure herein regarding an RGEN can likewise apply to the RGEN component of an RGEN-CPP complex, unless otherwise noted.

An RGEN herein that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas9 protein disclosed herein), or a variant thereof retaining some or all activity in each endonuclease domain of the Cas protein, is a suitable example of an RGEN that can cleave both strands of a DNA target sequence. A Cas9 protein comprising functional RuvC and HNH nuclease domains is an example of a Cas protein that can cleave both strands of a DNA target sequence. An RGEN herein that can cleave both strands of a DNA target sequence typically cuts both strands at the same position such that blunt-ends (i.e., no nucleotide overhangs) are formed at the cut site.

An RGEN herein that can cleave one strand of a DNA target sequence can be characterized herein as having nickase activity (e.g., partial cleaving capability). A Cas nickase (e.g., Cas9 nickase) herein typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain.

Non-limiting examples of Cas9 nickases suitable for use herein are disclosed by Gasiunas et al. (Proc. Natl. Acad. Sci. U.S.A. 109:E2579-E2586), Jinek et al. (Science 337:816-821), Sapranauskas et al. (Nucleic Acids Res. 39:9275-9282) and in U.S. Patent Appl. Publ. No. 2014/0189896, which are incorporated herein by reference. For example, a Cas9 nickase herein can comprise an S. thermophilus Cas9 having an Asp-31 substitution (e.g., Asp-31-Ala) (an example of a mutant RuvC domain), or a His-865 substitution (e.g., His-865-Ala), Asn-882 substitution (e.g., Asn-882-Ala), or Asn-891 substitution (e.g., Asn-891-Ala) (examples of mutant HNH domains). Also for example, a Cas9 nickase herein can comprise an S. pyogenes Cas9 having an Asp-10 substitution (e.g., Asp-10-Ala), Glu-762 substitution (e.g., Glu-762-Ala), or Asp-986 substitution (e.g., Asp-986-Ala) (examples of mutant RuvC domains), or a His-840 substitution (e.g., His-840-Ala), Asn-854 substitution (e.g., Asn-854-Ala), or Asn-863 substitution (e.g., Asn-863-Ala) (examples of mutant HNH domains). Regarding S. pyogenes Cas9, the three RuvC subdomains are generally located at amino acid residues 1-59, 718-769 and 909-1098, respectively, and the HNH domain is located at amino acid residues 775-908 (Nishimasu et al., Cell 156:935-949).

A Cas9 nickase herein can be used for various purposes in cells, if desired. For example, a Cas9 nickase can be used to stimulate HR at or near a DNA target site sequence with a suitable polynucleotide modification template. Since nicked DNA is not a substrate for NHEJ processes, but is recognized by HR processes, nicking DNA at a specific target site should render the site more receptive to HR with a suitable polynucleotide modification template.

As another example, a pair of Cas9 nickases can be used to increase the specificity of DNA targeting. In general, this can be done by providing two Cas9 nickases that, by virtue of being associated with RNA components with different guide sequences, target and nick nearby DNA sequences on opposite strands in the region for desired targeting. Such nearby cleavage of each DNA strand creates a DSB (i.e., a DSB with single-stranded overhangs), which is then recognized as a substrate for NHEJ (leading to indel formation) or HR (leading to recombination with a suitable polynucleotide modification template, if provided). Each nick in these embodiments can be at least about 5, 10, 15, 20, 30, 40, 50, 60, 70, 80, 90, or 100 (or any integer between 5 and 100) bases apart from each other, for example. One or two Cas9 nickase proteins herein can be used in a Cas9 nickase pair as described above. For example, a Cas9 nickase with a mutant RuvC domain, but functioning HNH domain (i.e., Cas9 HNH⁺/RuvC⁻), could be used (e.g., S. pyogenes Cas9 HNH⁺/RuvC⁻). Each Cas9 nickase (e.g., Cas9 HNH⁺/RuvC⁻) would be directed to specific DNA sites nearby each other (up to 100 base pairs apart) by using suitable RNA components herein with guide RNA sequences targeting each nickase to each specific DNA site.

An RGEN in certain embodiments can bind to a DNA target site sequence, but does not cleave any strand at the target site sequence. Such an RGEN may comprise a Cas protein in which all of its nuclease domains are mutant, dysfunctional. For example, a Cas9 protein herein that can bind to a DNA target site sequence, but does not cleave any strand at the target site sequence, may comprise both a mutant, dysfunctional RuvC domain and a mutant, dysfunctional HNH domain. Non-limiting examples of such a Cas9 protein comprise any of the RuvC and HNH nuclease domain mutations disclosed above (e.g., an S. pyogenes Cas9 with an Asp-10 substitution such as Asp-10-Ala and a His-840 substitution such as His-840-Ala). A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein). For example, a Cas9 comprising an S. pyogenes Cas9 with an Asp-10 substitution (e.g., Asp-10-Ala) and a His-840 substitution (e.g., His-840-Ala) can be fused to a VP16 or VP64 transcriptional activator domain. The guide sequence used in the RNA component of such an RGEN would be complementary to a DNA sequence in a gene promoter or other regulatory element (e.g., intron), for example.

An RGEN herein can bind to a target site sequence, and optionally cleave one or both strands of the target site sequence, in a chromosome, episome, or any other DNA molecule in the genome of a cell. This recognition and binding of a target sequence is specific, given that an RNA component of the RGEN comprises a sequence (guide sequence) that is complementary to a strand of the target sequence.

The terms “target site”, “target sequence”, “target DNA”, “DNA target sequence”, “target locus”, “protospacer” and the like are used interchangeably herein. A target site sequence refers to a polynucleotide sequence on a chromosome, episome, or any other DNA molecule in the genome of a cell to which an RGEN herein can recognize, bind to, and optionally nick or cleave. A target site can be (i) an endogenous/native site in the cell, (ii) heterologous to the cell and therefore not be naturally occurring in the genome, or (iii) found in a heterologous genomic location compared to where it natively occurs.

A target site sequence herein is at least 13 nucleotides in length and has a strand with sufficient complementarity to a variable targeting domain (of a crRNA or gRNA) to be capable of hybridizing with the guide sequence and direct sequence-specific binding of a Cas protein or Cas protein complex to the target sequence (if a suitable PAM is adjacent to the target sequence in certain embodiments). A cleavage/nick site (applicable with a endonucleolytic or nicking Cas) can be within the target sequence (e.g., using a Cas9) or a cleavage/nick site could be outside of the target sequence (e.g., using a Cas9 fused to a heterologous endonuclease domain such as one derived from a Fokl enzyme). It is also possible for a target site sequence to be bound by an RGEN lacking cleavage or nicking activity.

An “artificial target site” or “artificial target sequence” herein refers to a target sequence that has been introduced into the genome of a cell. An artificial target sequence in some embodiments can be identical in sequence to a native target sequence in the genome of the cell, but be located at a different position (a heterologous position) in the genome, or it can different from the native target sequence if located at the same position in the genome of the cell.

The length of a target sequence herein can be at least 13, 14, 15, 16, 17, 18, 19, 20, 21, 22, 23, 24, 25, 26, 27, 28, 29, or 30 nucleotides; between 13-30 nucleotides; between 17-25 nucleotides; or between 17-20 nucleotides, for example. This length can include or exclude a PAM (protospacer-adjacent motif) sequence. Also, a strand of a target sequence herein has sufficient complementarity with a variable targeting domain (of a crRNA or gRNA) to hybridize with the guide sequence and direct sequence-specific binding of a Cas protein or Cas protein complex to the target sequence (if a suitable PAM is adjacent to the target sequence, see below). The degree of complementarity between a guide sequence and a strand of its corresponding DNA target sequence is at least about 90%, 91%, 92%, 93%, 94%, 95%, 96%, 97%, 98%, 99%, or 100%, for example. A target site herein may be located in a sequence encoding a gene product (e.g., a protein or an RNA) or a non-coding sequence (e.g., a regulatory sequence or a “junk” sequence), for example.

A “protospacer adjacent motif” (PAM) herein refers to a short sequence that is recognized by an RGEN herein. The sequence and length of a PAM herein can differ depending on the Cas protein or Cas protein complex used, but are typically 2, 3, 4, 5, 6, 7, or 8 nucleotides long, for example.

A PAM (protospacer-adjacent motif) sequence may be adjacent to the target site sequence. A PAM sequence is a short DNA sequence recognized by an RGEN herein. The associated PAM and first 11 nucleotides of a DNA target sequence are likely important to Cas9/gRNA targeting and cleavage (Jiang et al., Nat. Biotech. 31:233-239). The length of a PAM sequence herein can vary depending on the Cas protein or Cas protein complex used, but is typically 2, 3, 4, 5, 6, 7, or 8 nucleotides long, for example. A PAM sequence is immediately downstream from, or within 2, or 3 nucleotides downstream of, a target site sequence that is complementary to the strand in the target site that is in turn complementary to an RNA component guide sequence, for example. In embodiments herein in which an RGEN is an endonucleolytically active Cas9 protein complexed with an RNA component, Cas9 binds to the target sequence as directed by the RNA component and cleaves both strands immediately 5′ of the third nucleotide position upstream of the PAM sequence. Consider the following example of a target site:PAM sequence: 5′-NNNNNNNNNNNNNNNNNNNNXGG-3′ (SEQ ID NO:52).

N can be A, C, T, or G, and X can be A, C, T, or G in this example sequence (X can also be referred to as N_{PAM}). The PAM sequence in this example is XGG (underlined). A suitable Cas9/RNA component complex would cleave this target immediately 5′ of the double-underlined N. The string of N's in SEQ ID NO:52 represents target sequence that is at least about 90%, 91° A, 92%, 93%, 94%, 95%, 96%, 97%, 98%, 99%, or 100% identical, for example, with a guide sequence in an RNA component herein (where any T′s of the DNA target sequence would align with any U's of the RNA guide sequence). A guide sequence of an RNA component of a Cas9 complex, in recognizing and binding at this target sequence (which is representive of target sites herein), would anneal with the complement sequence of the string of N's; the percent complementarity between a guide sequence and the target site complement is at least about 90%, 91%, 92%, 93%, 94%, 95%, 96%, 97%, 98%, 99%, or 100%, for example. If a Cas9 nickase is used to target SEQ ID NO:52 in a genome, the nickase would nick immediately 5′ of the double-underlined N or at the same position of the complementary strand, depending on which endonuclease domain in the nickase is dysfunctional. If a Cas9 having no nucleolytic activity (both RuvC and HNH domains dysfuntional) is used to target SEQ ID NO:52 in a genome, it would recognize and bind the target sequence, but not make any cuts to the sequence.

A PAM herein is typically selected in view of the type of RGEN being employed. A PAM sequence herein may be one recognized by an RGEN comprising a Cas, such as Cas9, derived from any of the species disclosed herein from which a Cas can be derived, for example. In certain embodiments, the PAM sequence may be one recognized by an RGEN comprising a Cas9 derived from S. pyogenes, S. thermophilus, S. agalactiae, N. meningitidis, T. denticola, or F. novicida. For example, a suitable Cas9 derived from S. pyogenes could be used to target genomic sequences having a PAM sequence of NGG (N can be A, C, T, or G). As other examples, a suitable Cas9 could be derived from any of the following species when targeting DNA sequences having the following PAM sequences: S. thermophilus (NNAGAA) S. agalactiae (NGG, NNAGAAW [W is A or T], NGGNG, N. meningitidis (NNNNGATT), T. denticola (NAAAAC), or F. novicida (NG) (where N's in all these particular PAM sequences are A, C, T, or G). Other examples of Cas9/PAMs useful herein include those disclosed in Shah et al. (RNA Biology 10:891-899) and Esvelt et al. (Nature Methods 10:1116-1121), which are incorporated herein by reference. Examples of target sequences herein follow SEQ ID NO:43, but with the ‘XGG’ PAM replaced by any one of the foregoing PAMs.

An RNA component herein can comprise a sequence complementary to a target site sequence in a chromosome or episome in a cell. An RGEN can specifically bind to a target site sequence, and optionally cleave one or both strands of the target site sequence, based on this sequence complementary. Thus, the complementary sequence of an RNA component in certain embodiments of the disclosed disclosure can also be referred to as a guide sequence or variable targeting domain.

The guide sequence of an RNA component (e.g., crRNA or gRNA) herein can be at least 13, 14, 15, 16, 17, 18, 19, 20, 21, 22, 23, 24, 25, 26, 27, 28, 29, or 30 ribonucleotides in length; between 13-30 ribonucleotides in length; between 17-25 ribonucleotides in length; or between 17-20 ribonucleotides in length, for example. In general, a guide sequence herein has sufficient complementarity with a strand of a target DNA sequence to hybridize with the target sequence and direct sequence-specific binding of a Cas protein or Cas protein complex to the target sequence (if a suitable PAM is adjacent to the target sequence). The degree of complementarity between a guide sequence and its corresponding DNA target sequence is at least about 90%, 91%, 92%, 93%, 94%, 95%, 96%, 97%, 98%, 99%, or 100%, for example. The guide sequence can be engineered accordingly to target an RGEN to a DNA target sequence in a cell.

An RNA component herein can comprise a crRNA, for example, which comprises a guide sequence and a repeat (tracrRNA mate) sequence. The guide sequence is typically located at or near (within 1, 2, 3, 4, 5, 6, 7, 8, 9, 10 or more bases) the 5′ end of the crRNA. Downstream the guide sequence of a crRNA is a “repeat” or “tracrRNA mate” sequence that is complementary to, and can hybridize with, sequence at the 5′ end of a tracrRNA. Guide and tracrRNA mate sequences can be immediately adjacent, or separated by 1, 2, 3, 4 or more bases, for example. A tracrRNA mate sequence has, for example, at least 50%, 60%, 70%, 80%, 90%, 95%, 96%, 97%, 98%, 99%, or 100% sequence complementarity to the 5′ end of a tracrRNA. In general, degree of complementarity can be with reference to the optimal alignment of the tracrRNA mate sequence and 5′ end of the tracrRNA sequence, along the length of the shorter of the two sequences. The length of a tracrRNA mate sequence herein can be at least 8, 9, 10, 11, 12, 13, 14, 15, 16, 17, or 18 ribonucleotides in length, for example, and hybridizes with sequence of the same or similar length (e.g., plus or minus 1, 2, 3, 4, or 5 bases) at the 5′ end of a tracrRNA. The length of a crRNA herein can be at least about 18, 20, 22, 24, 26, 28, 30, 32, 34, 36, 38, 40, 42, 44, 46, or 48 ribonucleotides; or about 18-48 ribonucleotides; or about 25-50 ribonucleotides, for example.

A tracrRNA can be included along with a crRNA in embodiments in which a Cas9 protein of a type II CRISPR system is comprised in the RGEN. A tracrRNA herein comprises in 5′-to-3′ direction (i) a sequence that anneals with the repeat region (tracrRNA mate sequence) of crRNA and (ii) a stem loop-containing portion. The length of a sequence of (i) can be the same as, or similar with (e.g., plus or minus 1, 2, 3, 4, or 5 bases), any of the tracrRNA mate sequence lengths disclosed above, for example. The total length of a tracrRNA herein (i.e., sequence components [i] and [ii]) can be at least about 30, 35, 40, 45, 50, 55, 60, 65, 70, 75, 80, 85, or 90 (or any integer between 30 and 90) ribonucleotides, for example. A tracrRNA may further include 1, 2, 3, 4, 5, or more uracil residues at the 3′-end, which may be present by virtue of expressing the tracrRNA with a transcription terminator sequence.

A tracrRNA herein can be derived from bacterial species, such as but not limited to Streptococcus species (e.g., S. pyogenes, S. thermophilus) or can include those disclosed in U.S. Pat. No. 8,697,359 and Chylinski et al. (RNA Biology 10:726-737), which are incorporated herein by reference.

The terms “ribozyme”, “ribonucleic acid enzyme” and “self-cleaving ribozyme” are used interchangeably herein. A ribozyme refers to one or more RNA sequences that form secondary, tertiary, and/or quaternary structure(s) that can cleave RNA at a specific site, particularly at a cis-site relative to the ribozyme sequence (i.e., auto-catalytic, or self-cleaving). The general nature of ribozyme nucleolytic activity has been described (e.g., Lilley, Biochem. Soc. Trans. 39:641-646). A “hammerhead ribozyme” (HHR) herein may comprise a small catalytic RNA motif made up of three base-paired stems and a core of highly conserved, non-complementary nucleotides that are involved in catalysis. Pley et al. (Nature 372:68-74) and Hammann et al. (RNA 18:871-885), which are incorporated herein by reference, disclose hammerhead ribozyme structure and activity. A hammerhead ribozyme herein may comprise a “minimal hammerhead” sequence as disclosed by Scott et al. (Cell 81:991-1002, incorporated herein by reference), for example.

The terms “targeting”, “gene targeting”, “DNA targeting”, “editing”, “gene editing” and “DNA editing” are used interchangeably herein. DNA targeting herein may be the specific introduction of an indel, knock-out, or knock-in at a particular DNA sequence, such as in a chromosome or episome of a cell. In general, DNA targeting can be performed herein by cleaving one or both strands at a specific DNA sequence in a cell with a Cas protein associated with a suitable RNA component. Such DNA cleavage, if a double-strand break (DSB), can prompt NHEJ processes which can lead to indel formation at the target site. Also, regardless of whether the cleavage is a single-strand break (SSB) or DSB, HR processes can be prompted if a suitable polynucleotide modification template or donor DNA is provided at the DNA nick or cleavage site. Such an HR process can be used to introduce a knock-out or knock-in at the target site, depending on the sequence of the polynucleotide modification template. Alternatively, DNA targeting herein can refer to specific association of a Cas/RNA component complex herein to a target DNA sequence, where the Cas protein does or does not cut a DNA strand (depending on the status of the Cas protein's endonucleolytic domains).

The term “indel” herein refers to an insertion or deletion of a nucleotide base or bases in a target DNA sequence in a chromosome or episome. Such an insertion or deletion may be of 1, 2, 3, 4, 5, 6, 7, 8, 9, 10, or more bases, for example. An indel in certain embodiments can be even larger, at least about 20, 30, 40, 50, 60, 70, 80, 90, or 100 bases. If an indel is introduced within an open reading frame (ORF) of a gene, oftentimes the indel disrupts wild type expression of protein encoded by the ORF by creating a frameshift mutation.

The terms “knock-out”, “gene knock-out” and “genetic knock-out” are used interchangeably herein. A knock-out represents a DNA sequence of a cell herein that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (by NHEJ, prompted by Cas-mediated cleavage), or by specific removal of sequence (by HR, prompted by Cas-mediated cleavage or nicking, when a suitable polynucleotide modification template is also used), that reduces or completely destroys the function of sequence at, adjoining, or near the targeting site. A knocked out DNA polynucleotide sequence herein can alternatively be characterized as being partially or totally disrupted or downregulated, for example.

The terms “knock-in”, “gene knock-in” and “genetic knock-in” are used interchangeably herein. A knock-in represents the replacement or insertion of a DNA sequence at a specific DNA sequence in a cell by targeting with a Cas protein (by HR, prompted by Cas-mediated cleavage or nicking, when a suitable donor DNA is also used). Examples of knock-ins are a specific insertion of a polynucleotide of interest, a heterologous amino acid coding sequence in a coding region of a gene, or a specific insertion of a transcriptional regulatory element in a genetic locus.

The terms “recombinant DNA molecule”, “recombinant construct”, “expression construct”, “ construct”, “construct”, and “recombinant DNA construct” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector or plasmid. The skilled artisan will also recognize that different independent gene editing events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “expression”, as used herein, refers to the production of a functional end-product (e.g., an mRNA, guide RNA, or a protein) in either precursor or mature form.

The term “providing” herein refers to providing (introducing) a nucleic acid (e.g., expression construct, plasmid) or protein into a cell. Providing includes reference to the incorporation of a nucleic acid into a eukaryotic or prokaryotic cell where the nucleic acid may be incorporated into the genome of the cell, and includes reference to the transient provision of a nucleic acid or protein to the cell. Providing includes reference to electroporation (Green M R, Sambrook J. 2012. Molecular Cloning: A Laboratory Manual, Fourth Edition ed. Cold Spring Harbor Laboratory Press, Cold Spring Harbor, N.Y.), heat-shock treatments (Green M R, Sambrook J. 2012. Molecular Cloning: A Laboratory Manual, Fourth Edition ed. Cold Spring Harbor Laboratory Press, Cold Spring Harbor, N.Y.), chemical treatments (Green M R, Sambrook J. 2012. Molecular Cloning: A Laboratory Manual, Fourth Edition ed. Cold Spring Harbor Laboratory Press, Cold Spring Harbor, N.Y.), phage delivery (Tyler B M, Goldberg R B. 1976. Transduction of chromosomal genes between enteric bacteria by bacteriophage P1. Journal of bacteriology 125:1105-1111), mating, conjugation and transduction (Methods for General and Molecular Bacteriology. 1994. ASM Press, Washington D.C.). Providing in the context of inserting a nucleic acid fragment (e.g., a recombinant DNA construct/expression construct) into a cell, includes “transfection” or “transformation” or “transduction” and includes reference to the incorporation of a nucleic acid fragment into a prokaryotic cell where the nucleic acid fragment may be incorporated into the genome of the cell (e.g., large circular genome, plasmid), converted into an autonomous replicon, or transiently expressed.

A nucleic acid molecule that has been provided into an organism/cell may be one that replicates autonomously in the organism/cell, or that integrates into the genome of the organism/cell, or that exists transiently in the cell without replicating or integrating. Non-limiting examples of nucleic acid molecules that can be provided to a cell are disclosed herein, such as plasmids and linear DNA molecules.

As described herein, the guide RNA/Cas endonuclease system can be used in combination with a co-delivered polynucleotide modification template to allow for editing of a genomic nucleotide sequence of interest. Also, as described herein, for each embodiment that uses a guide RNA/Cas endonuclease system, a similar guide polynucleotide/Cas endonuclease system can be deployed where the guide polynucleotide does not solely comprise ribonucleic acids but wherein the guide polynucleotide comprises a combination of RNA-DNA molecules or solely comprise DNA molecules.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” refers to a polynucleotide that comprises at least one nucleotide modification when compared to the nucleotide sequence to be edited. A nucleotide modification can include, for example: (i) a replacement of at least one nucleotide, (ii) a deletion of at least one nucleotide, (iii) an insertion of at least one nucleotide, or (iv) any combination of (i)-(iii). Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

As used herein, “donor DNA” is a DNA construct that comprises a polynucleotide of Interest to be inserted into the target site of a Cas endonuclease. The donor DNA construct can further comprise a first and a second region of homology that flank the polynucleotide of Interest. The first and second regions of homology of the donor DNA share homology to a first and a second genomic region, respectively, present in or flanking the target site of the plant genome.

A polynucleotide modification template or donor DNA can be able to undergo homologous recombination (HR) with a DNA target site. A “homologous sequence” within a polynucleotide modification template or donor DNA herein can, for example, comprise or consist of a sequence of at least about 25 nucleotides, for example, having 100% identity with a sequence at or near a target site, or at least about 90%, 91%, 92%, 93%, 94%, 95%, 96%, 97%, 98%, or 99% A identity with a sequence at or near a target site.

A polynucleotide modification template or donor DNA can have two homologous sequences separated by a sequence (or base pair) that is heterologous to sequence at a target site. These two homologous sequences of such a polynucleotide modification template or donor DNA can be referred to as “homology arms”, which flank the heterologous sequence. HR between a target site and a polynucleotide modification template or donor DNA with two homology arms typically results in the editing of a sequence at the target site.

A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a polynucleotide modification template or a Donor DNA can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bp. The amount of homology can also described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

In one embodiment, the disclosure describes a method for editing a nucleotide sequence in the genome of an Escherichia coli cell, the method comprising providing at least one recombinant DNA construct comprising a DNA sequence encoding a guide RNA and a circular polynucleotide modification template to an E.coli cell comprising a Cas9 endonuclease DNA sequence operably linked to an inducible promoter, wherein said Cas9 endonuclease DNA sequence encodes a Cas9 endonuclease that is capable of introducing a double-strand break at a target site in the genome of said E. coli cell, wherein said polynucleotide modification template comprises at least one nucleotide modification of said nucleotide sequence. The nucleotide sequence in the genome of an E. coli cell can be selected from the group consisting of a promoter sequence, a terminator sequence, a regulatory element sequence, a coding sequence, a prophage, a pseudogene, an exogenous gene, an endogenous gene. The recombinant DNA construct comprising a DNA sequence encoding a guide RNA can be provided via a circular plasmid. The recombinant DNA construct and the circular polynucleotide modification template can be provided on separate plasm ids or they can be provided on a single plasmid. The recombinant DNA construct and the circular polynucleotide template can be provided via one mean selected from the group consisting of electroporation, heat-shock, phage delivery, mating, conjugation and transduction, or any one combination thereof.

The nucleotide sequence to be edited can be a sequence that is endogenous, artificial, pre-existing, or transgenic to the cell that is being edited. For example, the nucleotide sequence in the genome of a cell can be a native gene, a mutated gene, a non-native gene, a foreign gene, or a transgene that is stably incorporated into the genome of a cell. Editing of such nucleotide may result in a further desired phenotype or genotype.

In one embodiment, the disclosure describes a method for editing a nucleotide sequence in the genome of an Escherichia coli cell, the method comprising providing at least a first recombinant DNA construct comprising a DNA sequence encoding a guide RNA, a circular polynucleotide modification template, and a second recombinant DNA construct comprising a DNA sequence encoding Cas9 endonuclease operably linked to an inducible promoter, to an E.coli cell, wherein the Cas9 endonuclease introduces a double-strand break at a target site in the genome of said E. coli cell, wherein said polynucleotide modification template comprises at least one nucleotide modification of said nucleotide sequence.

In one embodiment of the disclosure , the method comprises a method for inserting a polynucleotide sequence of interest in the genome of an Escherichia coli cell, the method comprising providing at least one recombinant DNA construct comprising a DNA sequence encoding a guide RNA and a circular donor DNA to an E.coli cell comprising a Cas9 endonuclease DNA sequence operably linked to an inducible promoter, wherein said Cas9 endonuclease DNA sequence encodes a Cas9 endonuclease that is capable of introducing a double-strand break at a target site in the genome of said E. coli cell, wherein said donor DNA comprises a polynucleotide.

Example of target sites in E. coli include sugar utilization genes (e.g. galactokinase, galK), metabolic genes (e.g. isocitrate dehydrogenase, icd, (Kabir M M, Shimizu K. 2004. Applied microbiology and biotechnology 65:84-96), biosynthetic genes (e.g. thymidylate synthase, thyA (Belfort M, Maley G, Pedersen-Lane J, Maley F. PNAS. 1983. 80(16):4914-18), transcriptional regulators (e.g. the general stress response regulator, rpoS (Notley-McRobb L, King T, Ferenci T (2002) J Bacteriol 184(3);806-11. PMID: 11790751), signaling proteins (e.g. sensor for anoxic redox control, arcB(Iuchi S, Matsuda Z, Fujiwara T, Lin E C (1990). Mol Microbiol 1990; 4(5);715-27. PMID: 2201868), tRNAs (e.g. tRNA alanine, alaU (Siekevitz P, Zamecnik PC (1981). Cell Biol 91(3 Pt 2);535-655. PMID: 7033244)), stress-response proteins (e.g. phage shock protein A, pspA (Adams H, Teertstra W, Demmers J, Boesten R, Tommassen J (2003). J Bacteriol 2003;185(4);1174-80. PMID: 12562786)), ribosomal components (e.g. S12 ribosoml protein, rpsL, (Funatsu G, Yaguchi M, Wittmann-Liebold B (1977). “Primary stucture of protein S12 from the small Escherichia coli ribosomal subunit.” FEBS Lett 73(1);12-7. PMID: 320034) and 23s ribosomal RNA, rrlD(Arkov A L, Hedenstierna K O, Murgola E J (2002). “Mutational evidence for a functional connection between two domains of 23S rRNA in translation termination.” J Bacteriol 184(18);5052-7. PMID: 12193621)), DNA replication (e.g. DNA polymerase II, polB (Chen H, Bryan S K, Moses R E (1989). “Cloning the polB gene of Escherichia coli and identification of its product.” J Biol Chem 264(34); 20591-5. PMID: 2684981)), Transcriptional machinery (e.g. the β′ subunit of RNA polymerase, rpoC (Squires C, Krainer A, Barry G, Shen W F, Squires C L (1981). “Nucleotide sequence at the end of the gene for the RNA polymerase beta′ subunit (rpoC).” Nucleic Acids Res 1981; 9(24); 6827-40. PMID: 6278450), transporters (eg. lactose permease, lacY(Buchel D E, Gronenborn B, Muller-Hill B (1980). “Sequence of the lactose permease gene.” Nature 1980; 283(5747);541-5. PMID: 6444453)), phage attachment sites (eg. λ attachment site, attB (Landy A, Ross W (1977). “Viral integration and excision: structure of the lambda att sites.” Science 197(4309);1147-60. PMID: 331474)), prophage genes (eg. rac prophoage inhibitor of cell division, kilR(Conter A, Bouche J P, Dassain M (1996). “Identification of a new inhibitor of essential division gene ftsZ as the kil gene of defective prophage Rac.” J Bacteriol 178(17);5100-4. PMID: 8752325)),or cell division (eg.cell division ring, ftsZ (Robinson A C, Kenan D J, Hatfull G F, Sullivan N F, Spiegelberg R, Donachie W D (1984). “DNA sequence and transcriptional organization of essential cell division genes ftsQ and ftsA of Escherichia coli: evidence for overlapping transcriptional units.” J Bacteriol 160(2);546-55. PMID: 6094474),). Additional genes suitable for target sites have been defined (Karp P D, Weaver D, Paley S, Fulcher C, Kubo A, Kothari A, Krummenacker M, Subhraveti P, Weerasinghe D, Gama-Castro S, Huerta A M, Muniz-Rascado L, Bonavides-Martinez C, Weiss V, Peralta-Gil M, Santos-Zavaleta A, Schroder I, Mackie A, Gunsalus R, Collado-Vides J, Keseler I M, Paulsen I. 2014. The EcoCyc Database. EcoSal Plus 2014; Keseler I M, Collado-Vides J, Santos-Zavaleta A, Peralta-Gil M, Gama-Castro S, Muniz-Rascado L, Bonavides-Martinez C, Paley S, Krummenacker M, Altman T, Kaipa P, Spaulding A, Pacheco J, Latendresse M, Fulcher C, Sarker M, Shearer AG, Mackie A, Paulsen I, Gunsalus R P, Karp P D. 2011. EcoCyc: a comprehensive database of Escherichia coli biology. Nucleic acids research 39:D583-590.; Keseler IM, Bonavides-Martinez C, Collado-Vides J, Gama-Castro S, Gunsalus R P, Johnson D A, Krummenacker M, Nolan L M, Paley S, Paulsen I T, Peralta-Gil M, Santos-Zavaleta A, Shearer A G, Karp P D. 2009. EcoCyc: a comprehensive view of Escherichia coli biology. Nucleic acids research 37:D464-470; Escherichia coli and Salmonella typhimurium: Cellular and Molecular Biology, 1987 First ed. American Society of Microbiology, Washington, D.C.

The terms “cell-penetrating peptide” (CPP) and “protein transduction domain” (PTD) are used interchangeably herein. A CPP refers to a peptide , typically of about 5-60 amino acid residues in length, that can facilitate cellular uptake of protein cargo, particularly one or more RGEN protein components described herein (e.g., Cas9 protein). Such protein cargo can be associated with one or more CPPs through covalent or non-covalent linkage. A CPP can also be characterized in certain embodiments as being able to facilitate the movement or traversal of protein cargo across/through one or more of a lipid bilayer, micelle, cell membrane, organelle membrane, vesicle membrane, or cell wall. A CPP herein can be cationic, amphipathic, or hydrophobic in certain embodiments. (see for example US provisional patent application NO. 62/036652, filed Aug. 13, 2014, incorporated by reference herein).

The terms “percent by volume”, “volume percent”, “vol %” and “v/v %” are used interchangeably herein. The percent by volume of a solute in a solution can be determined using the formula: [(volume of solute)/(volume of solution)]×100%.

The terms “percent by weight”, “weight percentage (wt %)” and “weight-weight percentage (% w/w)” are used interchangeably herein. Percent by weight refers to the percentage of a material on a mass basis as it is comprised in a composition, mixture, or solution.

The terms “polynucleotide”, “polynucleotide sequence”, and “nucleic acid sequence” are used interchangeably herein. These terms encompass nucleotide sequences and the like. A polynucleotide may be a polymer of DNA or RNA that is single- or double-stranded, that optionally contains synthetic, non-natural or altered nucleotide bases. A polynucleotide may be comprised of one or more segments of cDNA, genomic DNA, synthetic DNA, or mixtures thereof. Nucleotides (ribonucleotides or deoxyribonucleotides) can be referred to by a single letter designation as follows: “A” for adenylate or deoxyadenylate (for RNA or DNA, respectively), “C” for cytidylate or deoxycytidylate (for RNA or DNA, respectively), “G” for guanylate or deoxyguanylate (for RNA or DNA, respectively), “U” for uridylate (for RNA), “T” for deoxythymidylate (for DNA), “R” for purines (A or G), “Y” for pyrimidines (C or T), “K” for G or T, “H” for A or C or T, “I” for inosine, “W” for A or T, and “N” for any nucleotide (e.g., N can be A, C, T, or G, if referring to a DNA sequence; N can be A, C, U, or G, if referring to an RNA sequence). Any RNA sequence (e.g., crRNA, tracrRNA, gRNA) disclosed herein may be encoded by a suitable DNA sequence.

The term “isolated” refers to a polynucleotide or polypeptide molecule that has been completely or partially purified from its native source. In some instances, the isolated polynucleotide or polypeptide molecule is part of a greater composition, buffer system or reagent mix. For example, the isolated polynucleotide or polypeptide molecule can be comprised within a cell or organism in a heterologous manner.

The term “gene” refers to a DNA polynucleotide sequence that expresses an RNA (RNA is transcribed from the DNA polynucleotide sequence) from a coding region, which RNA can be a messenger RNA (encoding a protein) or a non-protein-coding RNA (e.g., a crRNA, tracrRNA, or gRNA herein). A gene may refer to the coding region alone, or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source, but arranged in a manner different than that found in nature. A “foreign” or “heterologous” gene refers to a gene that is introduced into the host organism by gene transfer. Foreign/heterologous genes can comprise native genes inserted into a non-native organism, native genes introduced into a new location within the native host, or chimeric genes. The polynucleotide sequences in certain embodiments disclosed herein are heterologous. A “codon-optimized” open reading frame has its frequency of codon usage designed to mimic the frequency of preferred codon usage of the host cell.

A “modified gene” or “edited gene” refers to a gene of interest that comprises at least one alteration when compared to its non-modified gene sequence. Such “alterations” include, for example: (i) replacement of at least one nucleotide, (ii) a deletion of at least one nucleotide, (iii) an insertion of at least one nucleotide, or (iv) any combination of (i)-(iii).

“Regulatory sequences” as used herein refer to nucleotide sequences located upstream of a gene's transcription start site (e.g., promoter), 5′ untranslated regions, and 3′ non-coding regions, and which may influence the transcription, processing or stability, or translation of an RNA transcribed from the gene. Regulatory sequences herein may include promoters, enhancers, silencers, 5′ untranslated leader sequences, introns, polyadenylation recognition sequences, RNA processing sites, effector binding sites, stem-loop structures, and other elements involved in regulation of gene expression. One or more regulatory elements herein may be heterologous to a coding region herein.

A “promoter” as used herein refers to a DNA sequence capable of controlling the transcription of RNA from a gene. In general, a promoter sequence is upstream of the transcription start site of a gene. Promoters may be derived in their entirety from a native gene, or be composed of different elements derived from different promoters found in nature, or even comprise synthetic DNA segments. Promoters that cause a gene to be expressed in a cell at most times under all circumstances are commonly referred to as “constitutive promoters”. One or more promoters herein may be heterologous to a coding region herein.

A “strong promoter” as used herein refers to a promoter that can direct a relatively large number of productive initiations per unit time, and/or is a promoter driving a higher level of gene transcription than the average transcription level of the genes in a cell.

Constitutive E. coli promoters are well known in the art and include promoters that lack regulation by a transcription factor and are recognized by RNA polymerase alone (Shimada T, Yamazaki Y, Tanaka K, Ishihama A. The whole set of constitutive promoters recognized by RNA polymerase RpoD holoenzyme of Escherichia coli. PLoS One. 2014. Mar. 6; 9(3):e90447; Science 2002, Stochastic Gene Expression in a Single Cell Vol. 297 no. 5584 pp. 1183-1186).

The terms “3′ non-coding sequence”, “transcription terminator” and “terminator” as used herein refer to DNA sequences located downstream of a coding sequence. This includes polyadenylation recognition sequences and other sequences encoding regulatory signals capable of affecting mRNA processing or gene expression.

The term “cassette” as used herein refers to a promoter operably linked to a DNA sequence encoding a protein-coding RNA or non-protein-coding RNA. A cassette may optionally be operably linked to a 3′ non-coding sequence.

The terms “upstream” and “downstream” as used herein with respect to polynucleotides refer to “5′ of” and “3′ of”, respectively.

The term “expression” as used herein refers to (i) transcription of RNA (e.g., mRNA or a non-protein coding RNA such as crRNA, tracrRNA, or gRNA) from a coding region, or (ii) translation of a polypeptide from mRNA.

When used to describe the expression of a gene or polynucleotide sequence, the terms “down-regulation”, “disruption”, “inhibition”, “inactivation”, and “silencing” are used interchangeably herein to refer to instances when the transcription of the polynucleotide sequence is reduced or eliminated. This results in the reduction or elimination of RNA transcripts from the polynucleotide sequence, which results in a reduction or elimination of protein expression derived from the polynucleotide sequence (if the gene comprised an ORF). Alternatively, down-regulation can refer to instances where protein translation from transcripts produced by the polynucleotide sequence is reduced or eliminated. Alternatively still, down-regulation can refer to instances where a protein expressed by the polynucleotide sequence has reduced activity. The reduction in any of the above processes (transcription, translation, protein activity) in a cell can be by about 20%, 30%, 40%, 50%, 60%, 70%, 80%, 90%, 95%, or 100% relative to the transcription, translation, or protein activity of a suitable control cell. Down-regulation can be the result of a targeting event as disclosed herein (e.g., indel, knock-out), for example.

The terms “control cell” and “suitable control cell” are used interchangeably herein and may be referenced with respect to a cell in which a particular modification (e.g., over-expression of a polynucleotide, down-regulation of a polynucleotide) has been made (i.e., an “experimental cell”). A control cell may be any cell that does not have or does not express the particular modification of the experimental cell. For example, a control cell may be a direct parent of the experimental cell, which direct parent cell does not have the particular modification that is in the experimental cell. Alternatively, a control cell may be a parent of the experimental cell that is removed by one or more generations. Alternatively still, a control cell may be a sibling of the experimental cell, which sibling does not comprise the particular modification that is present in the experimental cell.

The term “increased” as used herein may refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, and “improved” are used interchangeably herein. The term “increased” can be used to characterize the expression of a polynucleotide encoding a protein, for example, where “increased expression” can also mean “over-expression”.

The term “operably linked” as used herein refers to the association of two or more nucleic acid sequences such that that the function of one is affected by the other. For example, a promoter is operably linked with a coding sequence when it is capable of affecting the expression of that coding sequence. That is, the coding sequence is under the transcriptional control of the promoter. Coding sequences can be operably linked to regulatory sequences, for example. Also, for example, a crRNA can be operably linked (fused to) a tracrRNA herein such that the tracrRNA mate sequence of the crRNA anneals with 5′ sequence of the tracrRNA.

The term “recombinant” as used herein refers to an artificial combination of two otherwise separated segments of sequence, e.g., by chemical synthesis or by the manipulation of isolated segments of nucleic acids by genetic engineering techniques.

Methods for preparing recombinant constructs/vectors herein (e.g., a DNA polynucleotide encoding an RNA component cassette herein, or a DNA polynucleotide encoding a Cas protein or Cas-CPP fusion protein herein) can follow standard recombinant DNA and molecular cloning techniques as described by J. Sambrook and D. Russell (Molecular Cloning: A Laboratory Manual, 3rd Edition, Cold Spring Harbor Laboratory Press, Cold Spring Harbor, N.Y., 2001); T. J. Silhavy et al. (Experiments with Gene Fusions, Cold Spring Harbor Laboratory Press, Cold Spring Harbor, N.Y., 1984); and F. M. Ausubel et al. (Short Protocols in Molecular Biology, 5th Ed. Current Protocols, John Wiley and Sons, Inc., NY, 2002), for example.

A “phenotypic marker” is a screenable or selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers for E. coli include resistance to antibiotics (Ampicillin, Carbenicillin, Penicillin, Chloramphenicol, Kanamycin, Tetracycline, Eythromycin, spectinomycin, streptomycin) and auxotrophic markers (amino acid biosynthesis, sugar utilization, and vitamin biosynthesis) (Methods for General and Molecular Bacteriology. 1994. ASM Press, Washington D.C.).

Screenable markers in E. coli include fluorescent proteins (GFP, RFP, CFP, YFP), sugar utilization (lactose, ribose, glucose, sucrose, galactose, glycerol) (Methods for General and Molecular Bacteriology. 1994. ASM Press, Washington D.C.) and the generation of unique primer binding sites.

The terms “sequence identity” or “identity” as used herein with respect to polynucleotide or polypeptide sequences refer to the nucleic acid residues or amino acid residues in two sequences that are the same when aligned for maximum correspondence over a specified comparison window. Thus, “percentage of sequence identity” or “percent identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining percent complementarity of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

The Basic Local Alignment Search Tool (BLAST) algorithm, which is available online at the National Center for Biotechnology Information (NCBI) website, may be used, for example, to measure percent identity between or among two or more of the polynucleotide sequences (BLASTN algorithm) or polypeptide sequences (BLASTP algorithm) disclosed herein. Alternatively, percent identity between sequences may be performed using a Clustal algorithm (e.g., ClustalW or ClustalV). For multiple alignments using a Clustal method of alignment, the default values may correspond to GAP PENALTY=10 and GAP LENGTH PENALTY=10. Default parameters for pairwise alignments and calculation of percent identity of protein sequences using a Clustal method may be KTUPLE=1, GAP PENALTY=3, WINDOW=5 and DIAGONALS SAVED=5. For nucleic acids, these parameters may be KTUPLE=2, GAP PENALTY=5, WINDOW=4 and DIAGONALS SAVED=4. Alternatively still, percent identity between sequences may be performed using an EMBOSS algorithm (e.g., needle) with parameters such as GAP OPEN=10, GAP EXTEND=0.5, END GAP PENALTY=false, END GAP OPEN=10, END GAP EXTEND=0.5 using a BLOSUM matrix (e.g., BLOSUM62).

Herein, a first sequence that is “complementary” to a second sequence can alternatively be referred to as being in the “antisense” orientation with the second sequence.

Various polypeptide amino acid sequences and polynucleotide sequences are disclosed herein as features of certain embodiments of the disclosed disclosure. Variants of these sequences that are at least about 70-85%, 85-90%, or 90%-95% identical to the sequences disclosed herein can be used. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence.

All the amino acid residues disclosed herein at each amino acid position of Cas9 proteins herein are examples. Given that certain amino acids share similar structural and/or charge features with each other (i.e., conserved), the amino acid at each position in a Cas9 can be as provided in the disclosed sequences or substituted with a conserved amino acid residue (“conservative amino acid substitution”) as follows:
1. The following small aliphatic, nonpolar or slightly polar residues can substitute for each other: Ala (A), Ser (S), Thr (T), Pro (P), Gly (G);
2. The following polar, negatively charged residues and their amides can substitute for each other: Asp (D), Asn (N), Glu (E), Gln (Q);
3. The following polar, positively charged residues can substitute for each other: His (H), Arg (R), Lys (K);
4. The following aliphatic, nonpolar residues can substitute for each other: Ala (A), Leu (L), Ile (I), Val (V), Cys (C), Met (M); and
5. The following large aromatic residues can substitute for each other: Phe (F), Tyr (Y), Trp (W).

A genome of a bacterial cell, such as an E. coli cell, herein refers to a DNA molecule that can exist in a cell autonomously (can replicate and pass on to daughter cells). Genomic DNA can be either native or heterologous to a cell. Examples of genomic DNA in E. Coli include DNA located on a large circular DNA molecule as well as plasmid DNA

The term “cell” herein refers to any type of cell such as a prokaryotic or eukaryotic cell. A eukaryotic cell has a nucleus and other membrane-enclosed structures (organelles), whereas a prokaryotic cell lacks a nucleus. A cell in certain embodiments can be a mammalian cell or non-mammalian cell. Non-mammalian cells can be eukaryotic or prokaryotic. For example, a non-mammalian cell herein can refer to a microbial cell or cell of a non-mammalian multicellular organism such as a plant, insect, nematode, avian species, amphibian, reptile, or fish. A microbial cell herein can refer to a fungal cell (e.g., yeast cell), prokaryotic cell, protist cell (e.g., algal cell), euglenoid cell, stramenopile cell, or oomycete cell, for example. A prokaryotic cell herein can refer to a bacterial cell or archaeal cell, for example.

A bacterial cell can be those in the form of cocci, bacilli, spirochetes, spheroplasts, protoplasts, etc. Other non-limiting examples of bacteria include those that are Gram-negative and Gram-positive. Still other non-limiting examples of bacteria include those of the genera Salmonella (e.g., S. typhi, S. enteritidis), Shigella (e.g., S. dysenteriae), Escherichia (e.g., E. coli), Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus (e.g., S. aureus, S. epidermidis), Vibrio (e.g., V. cholerae), Aeromonas, Plessiomonas, Haemophilus (e.g., H. influenzae), Actinobacillus, Pasteurella, Mycoplasma (e.g., M. pneumonia), Ureaplasma, Rickettsia, Coxiella, Rochalimaea, Ehrlichia, Streptococcus (e.g., S. pyogenes, S. mutans, S. pneumoniae), Enterococcus (e.g., E. faecalis), Aerococcus, Gemella, Lactococcus (e.g., L. lactis), Leuconostoc (e.g., L. mesenteroides), Pedicoccus, Bacillus (e.g., B. cereus, B. subtilis, B. thuringiensis), Corynebacterium (e.g., C. diphtheriae), Arcanobacterium, Actinomyces, Rhodococcus, Listeria (e.g., L. monocytogenes), Erysipelothrix, Gardnerella, Neisseria (e.g., N. meningitidis, N. gonorrhoeae), Campylobacter, Arcobacter, Wolinella, Helicobacter (e.g., H. pylori), Achromobacter, Acinetobacter, Agrobacterium (e.g., A. tumefaciens), Alcaligenes, Chryseomonas, Comamonas, Eikenella, Flavimonas, Flavobacterium, Moraxella, Oligella, Pseudomonas (e.g., P. aeruginosa), Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus (e.g., L. lactis, L. acidophilus), Rothia, Clostridium (e.g., C. botulinum, C. perfringens), Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium (e.g., M. tuberculosis, M. bovis, M. leprae), Treponema, Borrelia (e.g., B. burgdorferi), Leptospira, and Chlamydiae. A bacteria can optionally be characterized as a pest/pathogen of a plant or animal (e.g., human) in certain embodiments. Bacteria can be comprised in a mixed microbial population (e.g., containing other bacteria, or containing yeast and/or other bacteria) in certain embodiments.

An archaeal cell in certain embodiments can be from any Archaeal phylum, such as Euryarchaeota, Crenarchaeota, Nanoarchaeota, Korarchaeota, Aigarchaeota, or Thaumarchaeota. Archaeal cells herein can be extremophilic (e.g., able to grow and/or thrive in physically or geochemically extreme conditions that are detrimental to most life), for example. Some examples of extremophilic archaea include those that are thermophilic (e.g., can grow at temperatures between 45-122° C.), hyperthermophilic (e.g., can grow at temperatures between 80-122° C.), acidophilic (e.g., can grow at pH levels of 3 or below), alkaliphilic (e.g., can grow at pH levels of 9 or above), and/or halophilic (e.g., can grow in high salt concentrations [e.g., 20-30% NaCl]). Examples of archaeal species include those of the genera Halobacterium (e.g., H. volcanii), Sulfolobus (e.g., S. solfataricus, S. acidocaldarius), Thermococcus (e.g., T. alcaliphilus, T. celer, T. chitonophagus, T. gammatolerans, T. hydrothermalis, T. kodakarensis, T. litoralis, T. peptonophilus, T. profundus, T. stetteri), Methanocaldococcus (e.g., M. thermolithotrophicus, M. jannaschii), Methanococcus (e.g., M. maripaludis), Methanothermobacter (e.g., M. marburgensis, M. thermautotrophicus), Archaeoglobus (e.g., A. fulgidus), Nitrosopumilus (e.g., N. maritimus), Metallosphaera (e.g., M. sedula), Ferroplasma, Thermoplasma, Methanobrevibacter (e.g., M. smithii), and Methanosphaera (e.g., M. stadtmanae).

Recombineering allows the editing of bacterial DNA using linear double and single stranded polynucleotide editing templates (Datsenko K A, Wanner B L. 2000. One-step inactivation of chromosomal genes in Escherichia coli K-12 using PCR products. Proceedings of the National Academy of Sciences of the United States of America 97:6640-6645; Thomason L C, Sawitzke J A, Li X, Costantino N, Court D L. 2014. Recombineering: genetic engineering in bacteria using homologous recombination. Current protocols in molecular biology/edited by Frederick M. Ausubel et al. 106:1 16 11-11 16 39). In order to utilize a linear or single stranded editing template expression of exogenous phage recombinase proteins are required (Datsenko K A, Wanner B L. 2000. One-step inactivation of chromosomal genes in Escherichia coli K-12 using PCR products. Proceedings of the National Academy of Sciences of the United States of America 97:6640-6645; U.S. Pat. No. 7,736,851 DNA cloning method, issued Jan. 15, 2010, incorporated by reference herein). Typically, small changes such as point mutations or deletions can be generated using short single stranded oligonucleotide editing templates. However, for larger changes or insertions of genes the presence of a selectable marker on the polynucleotide editing template is required in order to isolate colonies containing the desired edit due to the low frequency of recombination (ca 10⁻⁵ to 10⁻⁷). Once the edit is made the selectable marker must be removed, often leaving scar in the genome (Datsenko K A, Wanner B L. 2000. One-step inactivation of chromosomal genes in Escherichia coli K-12 using PCR products. Proceedings of the National Academy of Sciences of the United States of America 97:6640-6645).

Exogenous recombinase(s) includes proteins of homologous recombination systems provided in addition the cells native homologous recombination machinery (ie. expressed via non-natural means).

A RecET protein includes proteins of the ATP-independent, recA-indpendent homolgous recombination pathway of the Rac prophage (Kuzminov A. 1999. Recombinational repair of DNA damage in Escherichia coli and bacteriophage lambda. Microbiology and molecular biology reviews: MMBR 63:751-813).

A lambda-red protein includes a red, redβ, and redγ proteins of the phage lambda (Smith G R. 1988. Homologous recombination in procaryotes. Microbiological reviews 52:1-28).

A RecBCD inhibitor includes a protein that binds to and inhibits RecBCD function (eg lambda Gam protein) (Murphy K C. 2007. The lambda Gam protein inhibits RecBCD binding to dsDNA ends. Journal of molecular biology 371:19-24).

A DNA polynucleotide sequence comprising (i) a promoter operably linked to (ii) a nucleotide sequence encoding a guide RNA or a Cas endonuclease can typically be used for stable and/or transient expression of the guide RNA or cas endonuclease described herein. Such a polynucleotide sequence can be comprised within a plasmid, cosmid, phagemid, bacterial artificial chromosome (BAC), virus, or linear DNA (e.g., linear PCR product), for example, or any other type of vector or construct useful for providing a polynucleotide sequence into a cell.

Bacterial promoters include bacteriophage A promoter left (PL)(Menart V, Jevsevar S, Vilar M, Trobis A, Pavko A. 2003. Constitutive versus thermoinducible expression of heterologous proteins in Escherichia coli based on strong PR,PL promoters from phage lambda. Biotechnology and bioengineering 83:181-190), bacteriophage A promoter right (PR)(Menart V, Jevsevar S, Vilar M, Trobis A, Pavko A. 2003. Constitutive versus thermoinducible expression of heterologous proteins in Escherichia coli based on strong PR, PL promoters from phage lambda. Biotechnology and bioengineering 83:181-190,) the arabinose utilization operon promoter (PBAD)(Guzman L M, Belin D, Carson M J, Beckwith J. 1995. Tight regulation, modulation, and high-level expression by vectors containing the arabinose PBAD promoter. Journal of bacteriology 177:4121-4130), phage T7 RNA polymerase controlled promoters(PT7)(Ikeda R A, Ligman C M, Warshamana S. 1992. T7 promoter contacts essential for promoter activity in vivo. Nucleic acids research 20:2517-2524), the promoter of the lactose utilization operon of E. coli (Plac, (Gronenborn B. 1976. Overproduction of phage lambda repressor under control of the lac promotor of Escherichia coli. Molecular & general genetics: MGG 148:243-250) hybrid trp and lac promoters (Ptac)(de Boer H A, Comstock L J, Vasser M. 1983. The tac promoter: a functional hybrid derived from the trp and lac promoters. Proceedings of the National Academy of Sciences of the United States of America 80:21-25), and the phage T5 promoter (PT5)(Bujard H, Gentz R, Lanzer M, Stueber D, Mueller M, Ibrahimi I, Haeuptle M T, Dobberstein B. 1987. A T5 promoter-based transcription-translation system for the analysis of proteins in vitro and in vivo. Methods in enzymology 155:416-433). Other suitable promoters for expression in bacteria have been described (Green M R, Sambrook J. 2012. Molecular Clonine: A Laboratory Manual, Fourth Edition ed. Cold Spring Harbor Laboratory Press, Cold Spring Harbor, N.Y.; Karp P D, et al. 2014. The EcoCyc Database. EcoSal Plus 2014; Keseler I M et al. 2011. EcoCyc: a comprehensive database of Escherichia coli biology. Nucleic acids research 39:D583-590.)

In certain embodiments, a DNA polynucleotide comprising a cassette for expressing an RNA component comprises a suitable transcription termination sequence downstream of the RNA component sequence. Examples of transcription termination sequences useful herein are disclosed in U.S. Pat. Appl. Publ. No. 2014/0186906, which is herein incorporated by reference. Such embodiments typically comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more residues following the end of the RNA component sequence, depending on the choice of terminator sequence. These additional residues can be all U residues, or at least 90%, 91%, 92%, 93%, 94%, 95%, 96%, 97%, 98%, or 99% U residues, for example, depending on the choice of terminator sequence. Alternatively, a ribozyme sequence (e.g., hammerhead or HDV ribozyme) can be 3′ of (e.g., 1, 2, 3, 4, 5, 6, 7, 8, 9, 10 or more nucleotides downstream) the RNA component sequence, for example. A 3′ ribozyme sequence can be positioned accordingly such that it cleaves itself from the RNA component sequence; such cleavage would render a transcript ending exactly at the end of the RNA component sequence, or with 1, 2, 3, 4, 5, 6, 7, 8, 9, 10, 11, 12, 13, 14, 15, or more residues following the end of the RNA component sequence, for example.

An RGEN herein that can bind to a DNA target site sequence, but does not cleave any strand at the target site sequence, can be used in a DNA targeting method in other embodiments. Any RGEN disclosed herein that has only dysfunctional nuclease domains, but retains specific DNA-binding activity, can be used in this type of targeting method.

An RGEN linked or fused to an activator transcription factor or activator domain thereof can be used to up regulate expression of one or more polynucleotide sequences. A method incorporating such an activating RGEN can optionally be characterized as a transcriptional up-regulation or activation method. The level of transcriptional up-regulation in such a method can be at least about 25%, 50%, 75%, 100%, 250%, 500%, or 1000%, for example, compared to the transcription level before application of an activating RGEN.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an RGEN to a unique DNA target site. For example, two or more different RNA components can be used to prepare a mix of RGEN-CPP complexes in vitro (e.g., following a procedure disclosed herein for associating an RNA component with an RGEN protein-CPP complex), which mix is then contacted with a cell.

Another aspect of multiplex targeting herein can comprise providing two or more different RNA components in a cell which associate with the RGEN protein components of RGEN protein-CPP complexes that have traversed into the cell. Such a method can comprise, for example, providing to the cell (i) individual DNA polynucleotides, each of which express a particular RNA component that, and/or (ii) at least one DNA polynucleotide encoding two or more RNA components (e.g., see below disclosure regarding tandem ribozyme-RNA component cassettes).

A multiplex method can optionally target DNA sites very close to the same sequence (e.g., a promoter or open reading frame, and/or sites that are distant from each other (e.g., in different genes and/or chromosomes). A multiplex method in other embodiments can be performed with (for HR) or without (for NHEJ leading to indel and/or base substitution) suitable polynucleotide modification templates depending on the desired outcome of the targeting (if an endonuclease- or nickase-competent RGEN is used). In still other embodiments, a multiplex method can be performed with a repressing or activating RGEN as disclosed herein. For example, multiple repressing RGENs can be provided that down-regulate a set of genes, such as genes involved in a particular metabolic pathway.

Non-limiting examples of compositions and methods disclosed herein include:
1. A method for editing a nucleotide sequence in the genome of an Escherichia coli cell, the method comprising providing at least one recombinant DNA construct comprising a DNA sequence encoding a guide RNA and a circular polynucleotide modification template to an E.coli cell comprising a Cas9 endonuclease DNA sequence operably linked to an inducible promoter, wherein said Cas9 endonuclease DNA sequence encodes a Cas9 endonuclease that is capable of introducing a double-strand break at a target site in the genome of said E. coli cell, wherein said polynucleotide modification template comprises at least one nucleotide modification of said nucleotide sequence.
2. The method of embodiment 1, wherein the nucleotide sequence in the genome of an E. coli cell is selected from the group consisting of a promoter sequence, a terminator sequence, a regulatory element sequence, a coding sequence, a prophage, a pseudogene, an exogenous gene, and an endogenous gene.
3. The method of embodiment 1, wherein said recombinant DNA construct comprising a DNA sequence encoding a guide RNA is provided via a circular plasmid.
4. The method of embodiment 1, wherein the recombinant DNA construct and the circular polynucleotide modification template are each provided on separate plasmids.
5. The method of embodiment 1, wherein the recombinant DNA construct and the circular polynucleotide modification template are provided on a single plasmid.
6. The method of embodiment 1, wherein the recombinant DNA construct and the circular polynucleotide template are provided via one mean selected from the group consisting of electroporation, heat-shock, phage delivery mating, conjugation and transduction.
7. The method of embodiment 1, wherein said target site is flanked by a first genomic region and a second genomic region, wherein the circular polynucleotide template further comprises a first region of homology to said first genomic region and a second region of homology to said second genomic region.
8. The method of embodiment 1, wherein the E. coli cell does not express an exogenous recombinase protein.
9. The method of embodiment 1, wherein the E. coli cell does not express a protein selected from the group comprising a RecET protein, a lambda-red protein, and a RecBCD inhibitor.
10. The method of embodiment 1, further comprising growing progeny cells from said E. coli cell, wherein the progeny cell comprises the at least one nucleotide modification of said nucleotide sequence.
11. The method of embodiment 1 wherein the target site is located in an E. coli galK gene.
12. An E. coli cell produced by the method of embodiment 1.
13. An E. coli strain produced from the E. coli cell of embodiment 12.
14. A method for producing a galK mutant E. coli cell, the method comprising:
   a) providing at least one circular recombinant DNA construct comprising a DNA sequence encoding a guide RNA and at least one circular polynucleotide modification template to an E. coli cell comprising a Cas9 endonuclease DNA sequence operably linked to an inducible promoter, wherein said Cas9 endonuclease DNA sequence encodes a Cas endonuclease that is capable of introducing a double-strand break at a target site within a galK genomic sequence in the E. coli genome, wherein said circular polynucleotide modification template comprises at least one nucleotide modification of said galK genomic sequence;
   b) growing progeny cells from the E. coli cell of (a); and,
   c) evaluating the progeny cells of (b) for the presence of said at least one nucleotide modification.
15. A method for editing a nucleotide sequence in the genome of an Escherichia coli cell, the method comprising providing at least a first recombinant DNA construct comprising a DNA sequence encoding a guide RNA, a circular polynucleotide modification template, and a second recombinant DNA construct comprising a DNA sequence encoding Cas9 endonuclease operably linked to an inducible promoter, to an E. coli cell, wherein the Cas9 endonuclease introduces a double-strand break at a target site in the genome of said E. coli cell, wherein said polynucleotide modification template comprises at least one nucleotide modification of said nucleotide sequence.
16. The method of embodiment 15, wherein the first recombinant DNA construct, the second recombinant DNA construct, and the circular polynucleotide modification template are each provided on separate plasm ids.
17. The method of embodiment 1, wherein the first recombinant DNA construct, the second recombinant DNA construct, and the circular polynucleotide modification template are provided on a single plasmid

### EXAMPLES

The disclosed disclosure is further defined in the following Examples. It should be understood that these Examples, while indicating certain preferred aspects of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various uses and conditions.

### Example 1

### Construction of a Cas9 Endonuclease Expression Vector for use in Escherichia coli

In this example an inducible Cas9 expression vector for genome editing in Escherichia coli was constructed. Cas9 expression in response to an inducer was confirmed.

The Cas9 gene from Streptococcus pyrogenes M1 GAS SF370 (SEQ ID NO: 1) was Yarrowia codon optimized per standard techniques known in the art (SEQ ID NO: 2). In order to localize the Cas9 protein to the nucleus of the cells, Simian virus 40 (SV40) monopartite (MAPKKKRKV, SEQ ID NO: 3) nuclear localization signal was incorporated at the carboxy terminus of the Cas9 open reading frame. The Yarrowia codon optimized Cas9 gene was fused to a Yarrowia constitutive promoter, FBA1 (SEQ ID NO: 4), by standard molecular biology techniques. An example of a Yarrowia codon optimized Cas9 expression cassette (SEQ ID NO: 5) containing the constitutive FBA promoter, Yarrowia codon optimized Cas9, and the SV40 nuclear localization signal. The Cas9 expression cassette was cloned into the plasmid pZuf and the new construct called pZufCas9 (SEQ ID NO: 6).

The Yarrowia codon optimized Cas9-SV40 fusion gene (SEQ ID NO: 7) was amplified from pZufCas9 using standard molecular biology techniques. Primers for the reaction were GGGGGAATTCGACAAGAAATACTCCATCGGCCTGG (Forward, SEQ ID NO: 8) and CCCCAAGCTTAGCGGCCGCTTAGACCTTTCG (Reverse, SEQ ID NO: 9) adding a 5′ EcoRI site and a 3′ HindIII site to the fusion. The PCR product (SEQ ID NO: 10) was purified using standard techniques. The purified fragment was cloned into the EcoRI and Hindil sites of pBAD/HisB from life technologies (SEQ ID NO: 11) to create pRF48 (SEQ ID NO: 12).

E. coli Top10 cells (Life technologies) were transformed with pRF48. The transformed cells were maintained on L broth (1% (w/v) Tryptone, 0.5% (w/v) Yeast extract. 1% (w/v) NaCl)+100 μg/ml Ampicillin+0.4% (w/v) glucose to repress expression of the Cas9 protein. Cells were grown at 37° C. overnight at 220 RPM in L Broth+100 μg/ml Ampicillin+0.4%(w/v) glucose. The cells were diluted 1:100 in 1 L of 2× YT medium (1.6% Tryptone, 1.0% (w/v) Yeast Extract, 0.5%(w/v) NaCl) in a 2.8 L Fernbach flask. The culture was grown at 37° C. 220 RPM until OD₆₀₀ reached 0.438. 1 ml of culture was pelleted and resuspend in 43.8 μl of 1× Laemmli buffer and frozen at −20° C. L-arabinose was added to a final concentration of 0.2% (w/v) to induce the P_{BAD} promoter driving the Yarrowia optimized Cas9 gene. The culture was shifted to 18° C., 180 RPM for 20 hours.

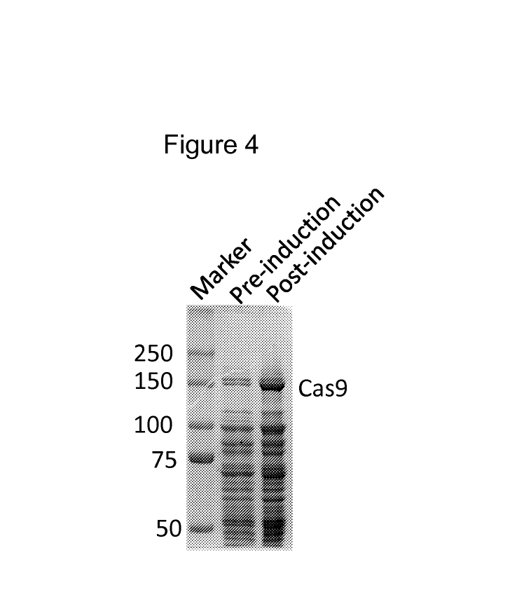

After induction by L-arabinose the OD₆₀₀ was 3.01. An aliquot of 0.332 ml of culture was pelleted. The cells were resuspended in 100 μl of 1× laemmeli buffer. Both the pre-induction and post induction samples were heated to 95° C. for 5 minutes and 10 μl was loaded onto a 12.5% tris-glycine SDS polyacrylamide gel. 200 volts were applied to the gel for 30 minutes. The gel was stained using simply blue stain to resolve protein bands. Expression of the Yarrowia optimized Cas9 protein in E. coli under control of an arabinose inducible promoter was robust (FIG. 4).

### Example 2

### Construction of Circular Expression Plasmids Encoding Single Guide RNAs Targeting the galK Gene of E. coli

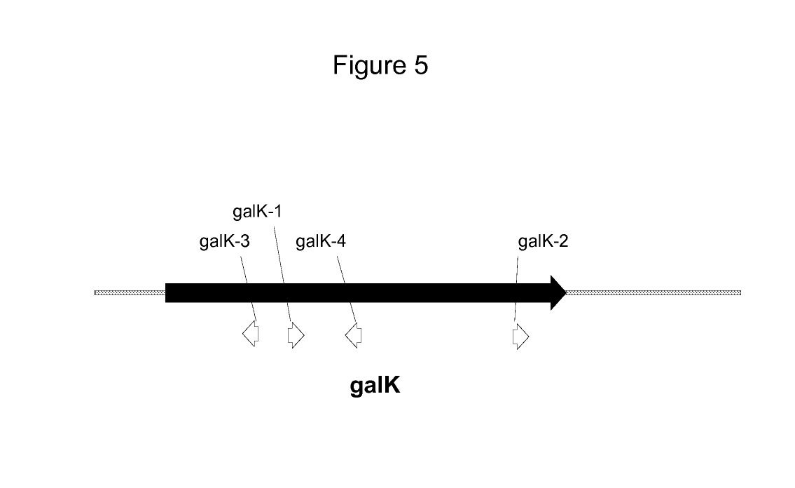

In order to modify (edit) the endogenous galK gene of E. coli four (4) Cas9 endonuclease target sites within the E. Coli galK gene were identified (FIG. 5): galK-1 (SEQ ID NO:13, table 1), galK-2 (SEQ ID NO: 14, table 1), galK-3 (SEQ ID NO: 15, table 1), and galK-4 (SEQ ID NO: 16, table 1).

TABLE 1
Targeting sequences for galK gene editing in
E. Coli.

| Targeting | | | | |
|---|---|---|---|---|
| sequence | | | | SEQ ID |
| name | Gene | Targeting sequence | PAM | No: |
| galK-1 | galK | ATCAGCGGCAATGTGCCGCA | GGG | 13 |
| galK-2 | galK | ATGACCGGCGGCGGATTTGG | CGG | 14 |
| galK-3 | galK | ATAGTTTTCATGTGCGACAA | TGG | 15 |
| galK-4 | galK | ATGATCTTTCTTGCCGAGCG | CGG | 16 |

DNA fragments corresponding to the genomic galK1 target sequences lacking the PAM domain (defined in Table 1) were fused to the a Streptococcus pyrogenes Cas Recognition domain (SEQ ID NO: 17) making complete DNA templates for single guide RNAs. The DNA fragments encoding the guide RNAs are show in SEQ ID NO: 18-21. The sgRNAs for gal1K-1 to gall -K4 are shown in SEQ ID Nos: 22-25.

In order to express the sgRNAs in E. coli cells four sgRNA expression cassettes were constructed (SEQ ID NOs: 28-31). The sgRNAs were put under control of the P_{L} promoter of the bacteriophage lambda (SEQ ID NO: 26). In order to induce transcriptional termination of the sgRNA the 3′ end of the CR domain was fused to the strong bacteriophage lambda terminator (SEQ ID NO: 27). The GalK-1 sgRNA expression cassette (SEQ ID NO: 28) was designed to target the galK-1 genomic target site (SEQ ID NO: 13). The GalK-2 sgRNA expression cassette (SEQ ID NO: 29) was designed to target the galK-1 genomic target site (SEQ ID NO:14). The GalK-3 sgRNA expression cassette (SEQ ID NO:30) was designed to target the galK-3 genomic target site (SEQ ID NO:15). The GalK-4 sgRNA expression cassette, (SEQ ID NO:31) was designed to target the galK-4 genomic target site (SEQ ID NO:16).

Each sgRNA expression cassette contained a 5′ HinDIII restriction site (AAGCTT) and a 3′ BamHI restriction site (GGATCC). Each sgRNA expression cassette was cloned into the HinDIII/BamHI sites of pACYC184 (SEQ ID NO: 32) to generate the circular plasmids (see guide RNA plasmid, FIG. 1 and FIG. 2) pRF50 (targeting galK-1, SEQ ID NO: 33), pRF51 (targeting galK-2, SEQ ID NO: 34), pRF53 (targeting galK-3, SEQ ID NO: 35), and pRF55 (targeting galK-4, SEQ ID NO: 36).

### Example 3

### Construction of Circular Plasmids Containing the Polynucleotide Modification Template for Gene Editing in E. coli

To enable gene editing (modification) in E. coli using (e/g/. gene deletion of the galK gene), a polynucleotide modification template was prepared that lacked a portion of the galK gene (referred to as the galK deletion template) as follows:

The 454 bp fragment directly 5′ of the translational start site of the E. coli galK gene (SEQ ID NO: 37) was amplified using standard PCR techniques using a forward primer (GGGaagcttggattatgttcagcgcgagc, SEQ ID NO: 38) adding a 5′ HinDIII restriction site for cloning and a reverse primer (tgccagtgcgggagtttcgtTTCTTACACTCCGGATTCGC, SEQ ID NO: 39) adding 20 bp of the sequencing directly 3′ of the stop codon of the galK gene to produce the upstream overlap extension product (SEQ ID NO: 40). The 376 bp directly 3′ of the translational stop site of the E. coli galK gene (SEQ ID NO: 41) was amplified using standard PCR techniques using forward primer (GCGAATCCGGAGTGTAAGAAacgaaactcccgcactggca, SEQ ID NO: 42) adding 20 bp of the sequence directly 5′ of the start codon of the galK gene and a reverse primer (GGGaagcttGCAAACAGCACCTGACGATCG, SEQ ID NO: 43) adding a 3′ HinDIII restriction site producing the downstream overlap extension product (SEQ ID NO: 44) The PCR products were purified using Zymo clean and concentrate columns. 10 ng of each PCR product were used to extend the overlapping 20nt using the forward primer for the 5′ fragment (GGGaagcttggattatgttcagcgcgagc, SEQ ID NO: 38) and the reverse primer of the 3′ fragment (GGGaagcttGCAAACAGCACCTGACGATCG, SEQ ID NO: 43). The full length galK deletion template (SEQ ID NO: 45) was cloned into the HinDIII sites of the conditionally replicating plasmid pKD3 (SEQ ID NO: 46) to create a circular galK deletion template plasmid pRF113 (SEQ ID NO: 47) The galK deletion template plasmid pRF113 (referred to as template plasmid in FIG. 1 and FIG. 2) lacks an expression cassette for a Pi protein (Inuzuka M. 1985. Plasmid-encoded initiation protein is required for activity at all three origins of plasmid R6K DNA replication in vitro. FEBS letters 181:236-240) thereby rendering it unable to replicate autonomously. Hence, once this circular template is provided to an E. coli cell it can function as a template for RGEN mediated gene editing but it will not be replicated and therefore be absent in any progeny cells that are cultured from said E. coli cell.

### Example 4

### Efficient Genome Editing of the galK Gene in E. coli using a Guide RNA/Cas Endonuclease System in Combination with a Circular Plasmid Containing a Polynucleotide Modification Template

Strain EF44 containing a deletion of the galE gene of E. coli is sensitive to the presence of galactose in the growth medium due to accumulation of the toxic product phosphor-galactose (Incorporate E. coli and S. typhimurium: Cellular and Molecular Biology Authors: Frederick C. Neidhardt, John L. Ingraham, Roy Curtiss III. ASM Press Washington D.C. 1987)). In this strain, mutations causing a loss of function in the gene encoding the galactose kinase (galK) rescue the galactose sensitivity allowing the strain to grow in the presence of galactose.

To create an E. coli strain containing a Cas9 plasmid comprising a Cas9 expression cassette (as depicted in FIG. 1), the plasmid pRF48 was introduced into the E. coli strain EF44 as follows. Strain EF44 strain was transformed with pRF48 (SEQ ID NO: 12) and colonies were selected on L broth agar plates containing 100 μg/ml Ampicillin and 0.4% (WN) glucose to repress the expression of the Cas9 gene from the pBAD promoter to create the E. coli strain EF56 (ΔgalE pRF48) containing the Cas9 plasmid.

A single colony of EF56 was inoculated in L Broth containing 100 μg/ml ampicillin and 0.4% (WN) Glucose and grown for 18 hours at 37° C. 230 RPM. The strain was then diluted into fresh L broth containing 100 μg/ml ampicillin and grown at 37° C. 230 RPM for 2 hours. L-arabinose was added to a final concentration of 0.2% (WN) to induce expression of Cas9 from the P_{BAD} promoter and the cells were grown for an additional 1 hour. Cells were made electrocompetent via standard protocols. 100 μl of induced electrocompetent EF56 cells were transformed with 200 ng of pACYC184 (SEQ ID NO: 32), pRF50 (SEQ ID NO: 33), pRF51 (SEQ ID NO: 34), pRF53 (SEQ ID NO: 35), or pRF55 (SEQ ID NO: 36) and either 1 μg pRF113 (SEQ ID NO: 47), 1 μg of linear polynucleotide modification template (SEQ ID NO: 44), or no polynucleotide modification template plasmid DNA. Cells were electroporated in a 1 mM gap cuvette at 1750 volts. 1 ml of SOC medium was added and cells were allowed to recover for 3 hours at 37° C. 230 RPM. Cells were plated on L broth plates solidified with 1.5% (w/v) agar containing 100 μg/ml Ampicillin and 25 μg/ml Chloramphenicol to select for cells containing both pRF48 (SEQ ID NO:12) and the corresponding pACYC184 (SEQ ID NO: 32), pRF50 (SEQ ID NO: 33), pRF51 (SEQ ID NO: 34), pRF53 (SEQ ID NO: 35), or pRF55 (SEQ ID NO: 36). Plates were incubated for 20 hours at 37° C.

Colonies were transferred from the L broth 100 μg/ml Ampicillin/25 μg/ml Chloramphenicol plates to Minimal A medium solidified with 1.5% (w/v) agar containing 0.2% (w/v) glycerol and 0.2% (w/v) galactose using replica plating to screen for galactose resistant isolates. For each transformation the frequency of galactose resistance was calculated by dividing the number of galactose resistant colonies by the total number of colonies on the original plate (Table 2).

TABLE 2
Frequency of Galactose resistant colonies.

| | | | Percent (%) |
|---|---|---|---|
| | | | Galactose |
| | | | resistant |
| | polynucleotide | Percent (%) Galactose | colonies from |
| | modification | resistant ± Standard | Homologous |
| gRNA plasmid | template | error of the mean | recombination |
| pACYC184 | None | 0 ± 0 | 0 |
| pACYC184 | pRF113 | 0 ± 0 | 0 |
| pACYC184 | Linear | 0 | 0 |
| pRF50 | None | 0.6 ± 0.4 | 0 |
| pRF50 | pRF113 | 06 ± 0.3 | 84.5 |
| pRF50 | Linear | 0.4 | 0 |
| pRF51 | None | 0.5 ± 0.5 | 0 |
| pRF51 | pRF113 | 0.2 ± 0.2 | 0 |
| pRF51 | Linear | 0.3 | 0 |
| pRF53 | None | 0.3 ± 0.2 | 0 |
| pRF53 | pRF113 | 3.6 ± 1.9 | 41.5 |
| pRF53 | Linear | 0 | 0 |
| pRF55 | None | 2.4 ± 1.5 | 0 |
| pRF55 | pRF113 | 1.9 ± 1.7 | 9 |
| pRF55 | Linear | 0.2 | 0 |

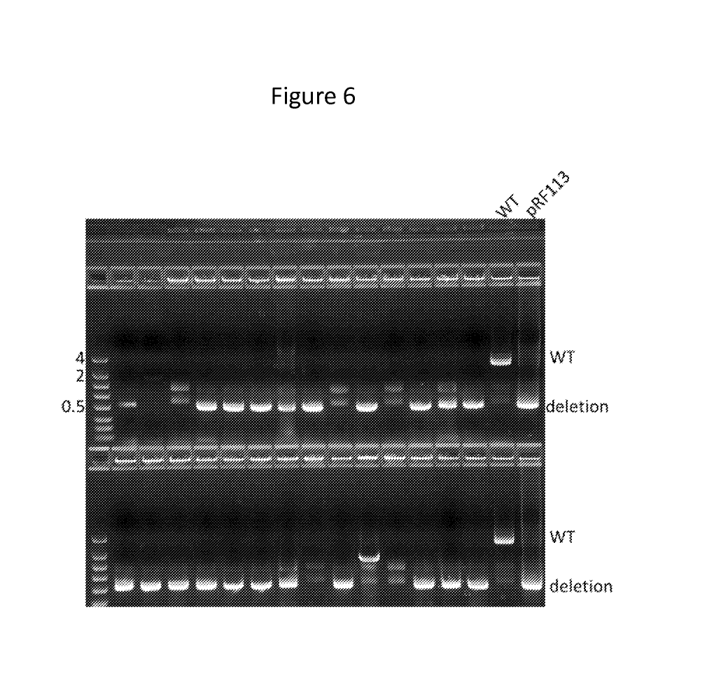

Frequencies were dependent on target site. In order to determine the frequency of Homologous recombination the galK locus (SEQ ID NO: 48) using standard PCR techniques using a forward primer (ggcgaagagaatcaacactgg, SEQ ID NO: 49) and a reverse primer (GCAAACAGCACCTGACGATCG, SEQ ID NO: 50). In a WT strain the entire galK locus is amplified (SEQ ID NO: 48) leading to a PCR product that is 1717 bp in length. In cells where recombination has occurred between the galK locus and the HR polynucleotide modification template pRF113 the PCR product is 569 bp in length (SEQ ID NO: 50). FIG. 6 shows a gel from the amplification of colonies from a pRF50/pRF113 editing experiment with an HR frequency of 75%. The HR frequency was determined by dividing the number of colonies where the deletion allele of galK was amplified, indicating precise editing by the total number of colonies assayed by colony PCR. Colonies that are Gal^{R} in the absence of polynucleotide modification template fail to allow amplification of the galK locus.

This example shows that efficient genome editing of the galK gene in E. coli was successfully accomplished using a guide RNA/Cas endonuclease system in combination with a circular plasmid containing a polynucleotide modification template.

That which is claimed:

1. A method for editing a nucleotide sequence in the genome of an Escherichia coli cell, the method comprising providing at least one recombinant DNA construct comprising a DNA sequence encoding a guide RNA and a circular polynucleotide modification template to an E.coli cell comprising a Cas9 endonuclease DNA sequence operably linked to an inducible promoter, wherein said Cas9 endonuclease DNA sequence encodes a Cas9 endonuclease that is capable of introducing a double-strand break at a target site in the genome of said E. coli cell, wherein said polynucleotide modification template comprises at least one nucleotide modification of said nucleotide sequence.

2. The method of claim 1, wherein the nucleotide sequence in the genome of an E. coli cell is selected from the group consisting of a promoter sequence, a terminator sequence, a regulatory element sequence, a coding sequence, a prophage, a pseudogene, and an exogenous gene.

3. The method of claim 1, wherein said recombinant DNA construct comprising a DNA sequence encoding a guide RNA is provided via a circular plasmid.

4. The method of claim 1, wherein the recombinant DNA construct and the circular polynucleotide modification template are each provided on separate plasm ids.

5. The method of claim 1, wherein the recombinant DNA construct and the circular polynucleotide modification template are provided on a single plasmid.

6. The method of claim 1, wherein the recombinant DNA construct and the circular polynucleotide template are provided via one mean selected from the group consisting of electroporation, heat-shock, phage delivery, mating, conjugation and transduction.

7. The method of claim 1, wherein said target site is flanked by a first genomic region and a second genomic region, wherein the circular polynucleotide template further comprises a first region of homology to said first genomic region and a second region of homology to said second genomic region.

8. The method of claim 1, wherein the E. coli cell does not express an exogenous recombinase protein.

9. The method of claim 1, wherein the E. coli cell does not express a protein selected from the group comprising a RecET protein, a lambda-red protein, and a RecBCD inhibitor.

10. The method of claim 1, further comprising growing progeny cells from said E. coli cell, wherein the progeny cell comprises the at least one nucleotide modification of said nucleotide sequence.

11. The method of claim 1 wherein the target site is located in an E. coli galK gene.

12. An E. coli cell produced by the method of claim 1.

13. An E. coli strain produced from the E. coli cell of claim 12.

14. A method for producing a galK mutant E. coli cell, the method comprising:
a) providing at least one circular recombinant DNA construct comprising a DNA sequence encoding a guide RNA and at least one circular polynucleotide modification template to an E. coli cell comprising a Cas9 endonuclease DNA sequence operably linked to an inducible promoter, wherein said Cas9 endonuclease DNA sequence encodes a Cas endonuclease that is capable of introducing a double-strand break at a target site within a galK genomic sequence in the E. coli genome, wherein said circular polynucleotide modification template comprises at least one nucleotide modification of said galK genomic sequence;
b) growing progeny cells from the E. coli cell of (a) ; and,
c) evaluating the progeny cells of (b) for the presence of said at least one nucleotide modification.

15. A method for editing a nucleotide sequence in the genome of an Escherichia coli cell, the method comprising providing at least a first recombinant DNA construct comprising a DNA sequence encoding a guide RNA, a circular polynucleotide modification template, and a second recombinant DNA construct comprising a DNA sequence encoding Cas9 endonuclease operably linked to an inducible promoter, to an E.coli cell, wherein the Cas9 endonuclease introduces a double-strand break at a target site in the genome of said E. coli cell, wherein said polynucleotide modification template comprises at least one nucleotide modification of said nucleotide sequence.

16. The method of claim 15, wherein the first recombinant DNA construct, the second recombinant DNA construct, and the circular polynucleotide modification template are each provided on separate plasm ids.

17. The method of claim 1, wherein the first recombinant DNA construct, the second recombinant DNA construct, and the circular polynucleotide modification template are provided on a single plasmid
